(19) United States
(12) Patent Application Publication
(10) Pub. No.: US 2026/0325091 A1
(43) Pub. Date: Oct. 22, 2026

(54) A Dental X-Ray Imaging System and a Method for Dental X-Ray Imaging of a Patient

(71) Applicant: PaloDEx Group Oy, Tuusula (FI)

(72) Inventors: Mika Pollari, Vantaa (FI); Markus Rintamäki, Tuusula (FI)

(21) Appl. No.: 19/115,599

(22) PCT Filed: Sep. 27, 2023
(86) PCT No.: PCT/FI2023/050510

§ 371 Date: Mar. 26, 2025

(30) Foreign Application Priority Data
Sep. 27, 2022 (FI) 20225840

(51) Int. Cl. A61B 6/51; A61B 6/00; A61B 6/10; A61B 6/46

(52) U.S. Cl. CPC A61B 6/51; A61B 6/102; A61B 6/4447; A61B 6/469; A61B 6/5247; A61B 6/542

(57) ABSTRACT

A dental X-ray imaging system for imaging a patient comprises a dental X-ray imaging unit and a control system. The dental X-ray imaging unit comprises: an X-ray source, an X-ray imaging detector, and a gantry comprising the source and the imaging detector. The control system is configured to: obtain at least one optical image of the patient; receive a scan request comprising region of interest (ROI) data; and define a ROI position based on the ROI data, the at least one optical image, dental atlas data, and at least one image analysis model formed based on previously collected reference image data. The invention relates also to a method a computer program, and a tangible non-volatile computer-readable medium for dental X-ray imaging of a patient.

### TECHNICAL FIELD

The invention concerns in general the technical field of dental X-ray imaging.

### BACKGROUND

Typically, the correct positioning of a patient may be one of the most time-consuming tasks of a user, e.g. an operator, of a dental X-ray imaging unit in a dental X-ray imaging process, but also one of the most important tasks. Traditionally, the patient may be positioned to the dental X-ray imaging unit using various supporting methods that are supposed to hold a head of the patient as stationary as possible.

Traditional supporting means may be a chin rest, a static bite stick, and a head support, where the forehead, temple, and/or back of the skull is supported. In addition, different kind of straps may be used to make the patient positioning as rigid as possible. In addition, some dental X-ray imaging units have such bite sticks that are attached to the dental X-ray imaging unit such that attachment means allow movements of the bite sticks in some directions.

One approach that can be considered as traditional as well is using scout images. This is a small dose panoramic image or a set of two projection images taken at 90 degrees angle that can be used as a targeting aid for a three-dimensional (3D) image.

A rigid setup is very important with this kind of approach. When the patient positioning (targeting) is done, the patient should keep steady for a whole imaging process. If the patient and/or the X-ray imaging unit moves, i.e. the position of the patient with relation to the X-ray imaging unit changes, between the positioning and the X-ray scanning phases, the resulting X-ray image might be diagnostically useless. Motion of the patient and/or the X-ray imaging unit during the scanning phase may cause severe artifacts in the resulting X-ray image and these artifacts caused by the motion needs to be corrected during a reconstruction of obtained image data during the scanning phase to the dental X-ray image, if possible or can be tried to be reduced by using a software, i.e. computer program,-based correction. The artifacts caused by the motion may affect the dental X-ray image quality significantly. The result may be e.g. a blurred image or a distorted image.

Moreover, typically in panoramic imaging anatomic shapes of the patient are unknown prior taking a panoramic image. Panoramic image quality is affected heavily based on how well a pre-defined imaging layer corresponds with the actual anatomic shapes, e.g. dental arch, of the patient. Typically, an average shape is used for all patients, which may lead to a non-optimized image quality. Furthermore, especially in the panoramic imaging the correct positioning of the patient is important. Incorrect positioning of the patient may lead to additional X-ray imaging of the patient.

Typically, the imaging workflow is manually controlled by the operator. Thus, the quality of the resulting dental X-ray image and the duration of the imaging may depend on the actions by the operator.

### SUMMARY

The following presents a simplified summary in order to provide basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

An objective of the invention is to present a dental X-ray imaging system, a method, a computer program, and a computer-readable medium for dental X-ray imaging of a patient, and a method, a computer program, and a computer-readable medium for determining aligned dental atlas data of a patient. Another objective of the invention is that the dental X-ray imaging system, the method, the computer program, and the computer-readable medium for dental X-ray imaging of a patient, and the method, the computer program, and the computer-readable medium for determining aligned dental atlas data of a patient improve quality of dental X-ray images.

The objectives of the invention are reached by a dental X-ray imaging system, methods, computer programs, and computer-readable mediums as defined by the respective independent claims.

According to a first aspect, a dental X-ray imaging system for dental X-ray imaging of a patient is provided, wherein the system comprises: a dental X-ray imaging unit comprising: an X-ray source part for emitting X-rays, an X-ray imaging detector part for receiving the X-rays from the source part, and a gantry part comprising the source part and the imaging detector part; and a control system configured to: obtain at least one optical image of the patient (600); receive a scan request comprising region of interest (ROI) data; and define a ROI position based on the ROI data, the at least one optical image, dental atlas data, and at least one image analysis model formed based on previously collected reference image data.

The at least one optical image of the patient may comprise at least one optical image where a dentition of the patient is at least partly visible, wherein the control system may be configured to: determine a plurality of head landmarks of the patient based on the at least one optical image of the patient and the at least one image analysis model, select a plurality of atlas landmarks corresponding to the plurality of head landmarks of the patient based on the dental atlas data, and register the plurality of atlas landmarks and the plurality of head landmarks of the patient to determine aligned dental atlas data of the patient.

The control system may be configured to define the ROI position based on the ROI data and the determined aligned dental atlas data of the patient.

Alternatively or in addition, the control system may further be configured to determine exposure parameters for the scan of the patient based on the imaging mode data further comprised in the scan request, the at least one optical image of the patient, and the at least one image analysis model.

The control system may be configured to: determine a plurality of head landmarks of the patient based on the at least one optical image of the patient and the at least one image analysis model, determine head size data of the patient based on the plurality of head landmarks of the patient, and use the head size data in the determination of the exposure parameters.

The control system may further be configured to: determine classification data of the patient based on the at least one optical image of the patient and the at least one image analysis model, and use the classification data in the determination of the exposure parameters.

Alternatively or in addition, the control system may further be configured to: determine at least one head landmark of the patient based on the at least one optical image of the patient and the at least one image analysis model, determine height data of the patient based on the at least one head landmark of the patient, and use the determined height data to adjust a height of the parts of the dental X-ray imaging unit.

Alternatively or in addition, the control system may further be configured to: determine a plurality of body landmarks of the patient based on the at least one optical image of the patient and the at least one image analysis model, determine width data of the patient based on the plurality of body landmarks of the patient, and use the determined width data to reduce a collision risk between the patient and the gantry part of the dental X-ray imaging unit.

The dental X-ray imaging system may comprise at least one optical imaging device configured to capture the at least one optical image of the patient (600). The ROI data may comprise an indication of at least one of the following: a single tooth, a range of teeth, a dental arch, both dental arches, a temporomandibular joint (TMJ), a whole dentition and the TMJs.

Alternatively or in addition, the control system may further be configured to produce patient position correction data for the patient positioning based on the at least one optical image of the patient and the at least one image analysis model. According to a second aspect, a method for dental imaging is provided, wherein the method is performed by an X-ray dental imaging system discussed above, wherein the method comprises: obtaining at least one optical image of the patient; receiving a scan request comprising region of interest (ROI) data; and defining a ROI position based on the ROI data, the at least one optical image, dental atlas data, and at least one image analysis model formed based on previously collected reference image data.

According to a third aspect, a computer program is provided, wherein the computer program comprises instructions which, when the program is executed by a computer, cause the computer to carry out the method discussed above.

According to a fourth aspect, a tangible non-volatile computer-readable medium is provided, wherein the computer-readable medium comprises instructions which, when executed by a computer, cause the computer to carry out the method discussed above.

According to a fifth aspect, a method for determining aligned dental atlas data of a patient is provided, wherein the method comprises: obtaining at least one optical image of the patient, where a dentition of the patient is at least partly visible; determining a plurality of head landmarks of the patient based on the at least one optical image of the patient and at least one image analysis model formed based on previously collected reference image data; selecting a plurality of atlas landmarks corresponding to the plurality of head landmarks of the patient based on dental atlas data; and registering the plurality of atlas landmarks and the plurality of head landmarks of the patient to determine the aligned dental atlas data of the patient.

According to a fifth aspect, a computer program is provided, wherein the computer program comprises instructions which, when the program is executed by a computer, cause the computer to carry out the method discussed above.

According to a sixth aspect, a tangible non-volatile computer-readable medium is provided, wherein the computer-readable medium comprises instructions which, when executed by a computer, cause the computer to carry out the method discussed above.

Various exemplifying and non-limiting embodiments of the invention both as to constructions and to methods of operation, together with additional objects and advantages thereof, will be best understood from the following description of specific exemplifying and non-limiting embodiments when read in connection with the accompanying drawings.

The verbs “to comprise” and “to include” are used in this document as open limitations that neither exclude nor require the existence of unrecited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

The embodiments of the invention are illustrated by way of example, and not by way of limitation, in the figures of the accompanying drawings.

FIG. 1 illustrates schematically an example of an example of a dental X-ray imaging system.

FIG. 2 illustrates schematically an example of a method for dental imaging of a patient.

FIG. 3 illustrates schematically an example of a reference imaging location for providing at least part of reference image data.

FIG. 4 illustrates schematically an example of a method for determining aligned dental atlas data of a patient.

FIGS. 5A and 5B illustrates schematically examples of a plurality of head landmarks of a patient.

FIG. 6A illustrates schematically an example of a method for defining coordinate transformation data.

FIG. 6B illustrates schematically an example of extracting device orientation data.

FIG. 6C illustrates schematically another example of extracting the device orientation data.

FIGS. 7A to 7D illustrate examples of a movement of a gantry part into a starting position of a scan trajectory.

FIG. 8A illustrates schematically an example of a method for determining exposure parameters for a scan of a patient.

FIG. 8B illustrates schematically yet another example of the plurality of head landmarks of the patient.

FIG. 9A illustrates schematically an example of a method for adjusting a height of parts of a dental X-ray imaging unit.

FIG. 9B illustrates schematically an example of a height off-set value.

FIG. 10A illustrates schematically an example of a method for reducing a collision risk between a patient and a gantry part of a dental X-ray imaging unit.

FIG. 10B illustrates schematically an example of a plurality of body landmarks of a patient.

FIG. 11 illustrates schematically an example of a control system of a dental X-ray imaging system.

FIG. 12 illustrates schematically an example of a method for detecting a patient readiness.

FIG. 13 illustrates schematically an example of a method for detecting a device readiness.

### DESCRIPTION OF THE EXEMPLIFYING EMBODIMENTS

In this description we use the following vocabulary concerning different phases of a dental X-ray imaging process. The term radiating means the phase comprising merely the irradiation, i.e. the phase when an X-ray source is providing an X-ray beam that travels through an object to an X-ray imaging detector. The object may be expected to remain as still, i.e. immobile, as possible during the radiating. During the radiating one or more parts of the dental X-ray imaging unit may move. The term scanning, in turn, means the phase comprising the radiating and moving of one or more parts of the dental X-ray imaging unit. The scanning does not comprise positioning of one or more parts of the X-ray imaging unit in a correct place for providing X-ray images. The term imaging means the whole process comprising radiating, scanning and positioning.

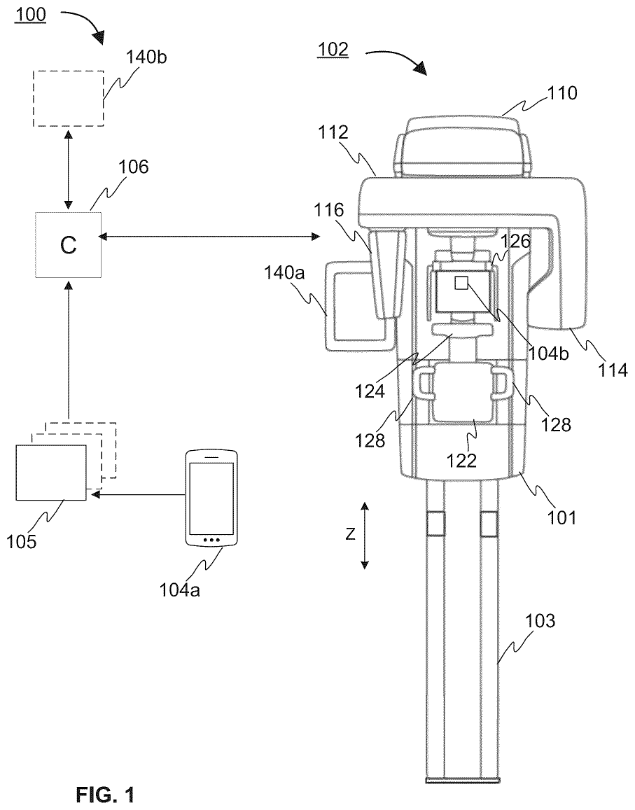

FIG. 1 illustrates an example of a dental X-ray imaging system 100 for dental X-ray imaging of a patient 600 (for sake of clarity the patient 600 is not shown in FIG. 1). The imaging system 100 comprises a dental X-ray imaging unit 102 for acquiring X-ray image data from an object, e.g. a patient or calibration target, in dental X-ray imaging, e.g. in extraoral dental X-ray imaging. The acquired X-ray image data is used to form a two-dimensional (2D) X-ray image or to recon-struct a three-dimensional (3D) X-ray volume from at least part of imaged object. The dental X-ray imaging system 100 further comprises a control system 106. The control system 106 may be electrically and/or communicatively coupled to the dental X-ray imaging unit 102. The implementation of the control system 106 may be done as a stand-alone unit or as a distributed control environment between a plurality of stand-alone units providing distributed controlling resource. Preferably, the computing unit 106 may be an embedded computer. The control system 106 may for example comprise a control unit of the dental X-ray imaging unit 102. The control system 106 may further comprise a computing unit of at least one imaging device 104a, 104b of the dental X-ray imaging system 100 and/or a computing unit being external to the dental X-ray imaging unit 102. The control unit of the dental X-ray imaging unit 102 is configured to control the operation of the dental X-ray imaging unit 102 at least in part. The control unit of the dental X-ray imaging unit 102 may be located proximate to the dental X-ray imaging unit 102 or the control unit of the dental X-ray imaging unit 102 may be embedded withing the dental X-ray imaging unit 102.

The dental X-ray imaging unit 102 may be configured to perform different types of imaging procedures (i.e. imaging modes), including, but not limited to computed tomography (CT) imaging and/or panoramic imaging. The CT imaging may be a cone beam CT (CBCT) imaging, wherein the beam is a cone-shaped beam, or other type of CT imaging for example, wherein the beam is a pyramidal-shaped beam, half-moon-shaped cone beam, or any other shaped beam. The CT imaging results (i.e. produces) the X-ray image data for the reconstruction of 3D volume from the at least part of the imaged object. The panoramic imaging may for example be standard panoramic imaging, pediatric panoramic imaging, orthozone panoramic imaging, wide arch panoramic imaging, orthogonal panoramic imaging or the like. The panoramic imaging results the X-ray image data for the formation of panoramic 2D image. Alternatively or in addition, the dental X-ray imaging unit 102 may be configured to perform cephalometric imaging, if the dental X-ray imaging unit 102 is equipped with parts, which are necessary for the cephalometric imaging. The cephalometric imaging may for example be cephalo pediatric lateral projection, cephalo lateral projection, cephalo posterior-anterior, and/or the like. The cephalometric imaging results the X-ray image data for the formation of cephalometric 2D image. FIG. 1 illustrates only one example of a dental X-ray imaging unit 102 for use with the concepts in the present disclosure.

The dental X-ray imaging unit 102 comprises a carriage part 101 that may be moveably supported on a support column 103. The carriage part 101 may be moved up and down in a height direction (Z), i.e. the vertical direction, by means of a guide motor (not shown in FIG. 1) that is configured to move the carriage 101 up and down along the supporting column 103 in the height direction. An upper shelf, 110 is configured to support a gantry part, i.e. a rotating part, 112, which is rotatable in a horizontal plane with respect to the upper shelf 110. The upper shelf 110 and/or gantry part 112 may comprise a rotating motor (not shown in FIG. 1) configured to rotate the gantry part 112. Alternatively or in addition, the upper shelf 110 may comprise a pivot motor (not shown in FIG. 1) configured to pivot the upper shelf 110 around the column 103. Alternatively or in addition, the dental X-ray imaging unit 102 may be mounted to a supporting structure (not shown in FIG. 1) exemplarily a wall to being supported by the column 103.

The dental X-ray imaging unit 102 comprises further an X-ray source part 114 and an X-ray imaging detector part 116, which are used in the acquisition of the X-ray image data. The gantry part 112 embodies and supports the source part 114 and the imaging detector part 116. The gantry part 112 may have substantially a form of letter C, as presented in FIG. 1, whereupon the source part 114 may be attached on one end of the gantry part 112 and the imaging detector part 116 may be attached on the other end of the gantry part 112 so that the source part 114 and the imaging detector part 116 are opposed from each other. The X-ray source part 114 comprises an X-ray source that emits X-rays (i.e. generates the X-ray beam) through the object being imaged, e.g. a head of the patient 600, to the X-ray imaging detector part 116, which comprises at least one X-ray detector that receives the emitted X-rays from the source part 114. The X-ray imaging detector part 116 further generates the X-ray image data from the X-ray exposed, i.e. imaged, object.

The X-ray imaging unit also comprises a collimator (not shown in FIG. 1) for the X-ray source part 114 to restrict and/or shape the beam of X-rays. The X-rays pass through a portion of the object, for example the patient's anatomy, e.g. patient's head. The anatomical structures through which the X-rays pass may absorb varying amounts of the X-ray energy. After passing through the object, the attenuated X-rays are received by the X-ray imaging detector part 116. The X-ray imaging detector part 116 is configured to convert the magnitude of the received X-ray energy and to produce a digitized output, i.e. the X-ray image data, representative of the unabsorbed X-rays at the at least one X-ray detector. The collection of digitized outputs from the X-ray imaging detector part 116 that correspond to a single emission of a beam of X-rays from the X-ray source part 114 may be referred to a projection image of the object being imaged, for example the head of the patient 600.

Furthermore, the dental X-ray imaging unit 102 may comprise patient support parts 124, 126 (as presented in FIG. 1, but not necessarily) that may be used for supporting the patient 600 in the CT or panoramic imaging. The patient support parts 124, 126 may comprise a chin support part 124 and/or a head support part 126. The chin support part 124 may support a tip of a chin of patient 600 and the head support part 126 may support a forehead or temple of the patient 600. The dental X-ray imaging unit 102 may comprise a lower shelf 122 that extends from the carriage 101. The lower shelf 122 may comprise the chin support part 124 as in the example dental X-ray imaging unit 102 of FIG. 1. The head support part 126 may extend from the upper shelf 110 through the rotating part 112 as in the example dental X-ray imaging unit 102 of FIG. 1. Alternatively, the lower shelf 122 may also comprise the head support part 126. The patient support parts, i.e. the chin support part 124 and/or the head support part 126, may be optional, and positioning of the patient 600 may be carried out in other manners. The dental X-ray imaging unit 102 may further comprise handles 128 for the patient 600 to grasp.

The gantry part 112 may be rotated by a rotating motor, for example. The rotation of the gantry part 112 rotates the X-ray source part 114 and the X-ray imaging detector part 116 around the object to be imaged, for example around a rotation axis along a scan trajectory. As the X-ray source part 114 and the X-ray imaging detector part 116 are rotated around the object, for example the head of the patient 600, the X-ray imaging device 102 operates to acquire a plurality of projection images of the object taken at incremental angles of rotation. The dental X-ray image may be formed from the plurality of projection images by reconstructing the X-ray image data to the dental X-ray image.

The dental X-ray imaging system 100 may further comprise at least one optical imaging device 104a, 104b. The at least one optical imaging device 104a, 104b may comprise at least one external imaging device 104a and/or at least one internal imaging device 104b. The at least one external imaging device 104a is external to the dental X-ray imaging unit 102. In other words, the at least one external imaging device 104a may be a device that is not part of the dental X-ray imaging unit 102. The use of the at least one external imaging device 104a may enable better lighting environment for collecting the optical image data in comparison to the at least one internal imaging device 104b, because at least some parts of the dental X-ray imaging unit 102 (e.g. the patient support parts 124, 126, the X-ray source part 114, and/or the X-ray imaging detector part 116) may disturb the lighting environment for capturing the optical images by using the at least one internal imaging device 104b. Alternatively or in addition, capturing the optical images by using the at least one external imaging device 104a is easy and simple. The at least one internal imaging device 104b may be arranged in connection with the dental X-ray imaging unit 102, e.g. embedded within the dental X-ray imaging unit 102. For example, the at least one internal imaging device 104b may be mounted (e.g. fixed) to the dental X-ray imaging unit 102. FIG. 1 illustrates only one non-limiting implementation example of the at least one internal imaging device 104b and any other number of internal imaging devices 104b may be used and located any other location on the dental X-ray imaging unit 102. The at least one imaging device 104a, 104b may be a 2D imaging device, and/or a 3D imaging device. Preferably, the at least one imaging device 104a, 104b is the 3D imaging device, i.e. a range imaging device. The 3D imaging device may be based on any 3D imaging technology, e.g. a stereo triangulation, a sheet of light triangulation, a structured light, a time-of-light, an interferometry, a code aperture and/or any other 3D imaging technology. The 3D imaging device may be a self-contained unit comprising all hardware and software related to the 3D imaging and possibly an internal computing unit inside the same module. Alternatively, the 3D imaging device may be a collection of separate components arranged in a 3D imaging configuration, wherein for example two optical cameras (e.g. a camera pair) are arranged in a stereovision configuration and 3D computation is done at an external computing unit, e.g. a control unit or similar. The 2D imaging device may for example be an optical camera and/or any other 2D imaging device. According to a non-limiting example, the at least one external imaging device 104a may be, but is not limited to, an optical camera, a mobile device comprising at least one optical camera (e.g. a mobile phone, a tablet computer), and/or standalone 3D face scanner system. The at least one internal optical imaging device 104b may be multifunctional optical imaging device. In other words, the at least one internal optical imaging device 104b may also be used in one or more other operations or functions, for example, but not limited to, patient positioning, motion detection, and/or motion correction, etc.

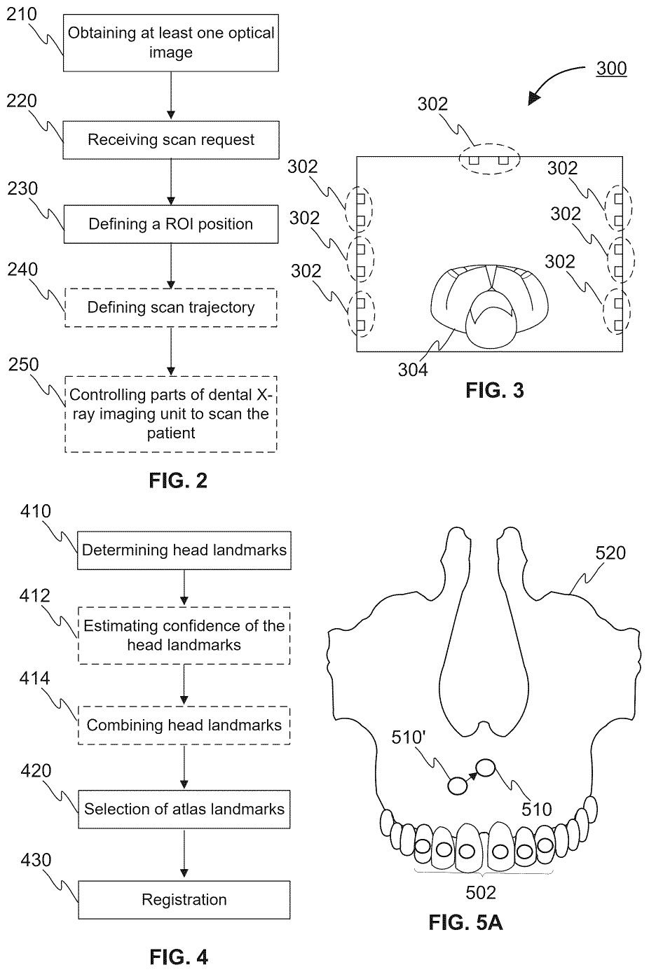

Next at least some example aspects of a method for dental imaging of the patient 600 are defined referring to FIG. 2. FIG. 2 illustrates the method as a flow chart. The method is performed by the dental X-ray system 100 discussed above.

At as step 210, the control system 106 obtains at least one optical image 105 of the patient 600. The at least one optical image 105 of the patient 600 may be captured by using the at least one optical imaging device 104a, 104b of the dental X-ray system 102, e.g. the at least one external imaging device 104a and/or the at least one internal imaging device 104b. The control system 106 may obtain the at least one optical image 105 of the patient 600 from the at least one optical imaging device 104a, 104b, e.g. from the at least one external imaging device 104a and/or from the at least one internal imaging device 104b. Alternatively, the control system 106 may obtain the at least one optical image of the patient 600 from a database into which the at least one optical image 105 captured by using the at least one optical imaging device 104a, 104b may be stored. In the example of FIG. 1, a non-limiting example is illustrated, wherein the obtained at least one optical image 105 is captured by the at least one external optical imaging device 104a, but alternatively or in addition, the obtained at least one optical image 105 may be captured by the at least one internal optical imaging device 104b. According to a non-limiting example, at least one optical image 105 of the patient 600 belonging to the at least one optical image 105 of the patient 600 may be captured when the patient 600 enters the room in which the dental X-ray imaging unit 102 is located. According to another non-limiting example, at least one optical image 105 of the patient 600 may be captured already before the patient 600 enters into the room in which the dental X-ray imaging unit 102 is located, as long as the at least one optical image of the patient 600 is substantially recently captured. The at least one optical image 105 may be a still image and/or a video image. The at least one optical image 105 of the patient 600 may comprise at least one facial image of the patient 600, at least one upper torso image of the patient 600, and/or at least one full body image of the patient 600.

At a step 220, the control system 106 receives a scan request. The scan request may be received locally or remotely via a user interface part 140a, 140b. The user interface part 140a, 140b may for example be located at the same location as the X-ray imaging unit 102 is located, i.e. the scan request may be received locally. For example, the X-ray imaging unit 102 may comprise the user interface part 140a, e.g. a touch screen, as in the example dental X-ray imaging unit 102 of FIG. 1. An operator of the X-ray imaging unit 102 may input the scan request via the user interface part 140a. Alternatively or in addition, the user interface part 140b may for example be located at another location than the location where the X-ray imaging unit 102 is located, i.e. the user interface part 140b may be located remotely from the X-ray imaging unit 102, and be communicatively coupled with the control system 106. In that case the scan request may be received remotely. The scan request comprises region of interest (ROI) data. The scan request may further comprise imaging mode data. The image mode data may for example comprise an indication of the imaging mode (i.e. the imaging modality) for the scan, e.g. CT imaging and possibly also the type of the CT imaging, panoramic imaging and possibly also the type of the panoramic imaging, or cephalometric imaging and possibly also the type of the cephalometric imaging. The ROI data may comprise an indication of one or more regions (e.g. one or more target anatomical structures) of the patient to be included in the scan, i.e. a target ROI. For example, the ROI data may comprise, but is not limited to, an indication of at least one of the following: a single tooth, a range of teeth, a dental arch (either maxillary or mandibular), both dental arches (maxillary and mandibular), a temporomandibular joint (TMJ), both TMJs, a whole dentition and TMJs. The scan request may further comprise patient identification data (for example, a patient name, a patient identification number, photograph, fingerprint, retinal scan, facial recognition, and/or other biometric data, etc.) and/or resolution data including an indication of a resolution to be used in the scan. In FIG. 2 the step 210 is presented before the step 220, but the step 220 may also be performed before or simultaneously with the step 210. At a step 230, the control system 106 defines a ROI position on the patient 600 based on the ROI data, the at least one optical image 105, dental atlas data 1116, and at least one image analysis model 1118. The ROI position on the patient 600 to be defined may depend on the imaging mode. For example, in case of the CT imaging the ROI position to be defined may be a position of a field-of-view (FOV). For example, in case of the panoramic imaging the ROI position to be defined may be a position of an imaging layer.

The dental atlas data 1116 may comprise atlas anatomy data. The anatomy data of the dental atlas data 1116 may for example comprise atlas anatomic structure data. The atlas anatomic structure data may for example comprise a location of one or more anatomic structures, e.g. teeth, the dental arch (maxillary and/or mandibular), the TMJ(s), a mandibular head(s), a chin menton, a chin notch, an acanthion, a tragus(es), an external auditory canal(s), a sinus(es), an eye socket(s) (e.g. a lower orbit of the eye socket), and/or any other anatomically relevant structure. The atlas anatomy data may further comprise atlas landmark data. The atlas landmark data may comprise a location of a plurality of atlas landmarks, e.g. a plurality of mandibular atlas landmarks, a plurality of maxillary atlas landmarks, and/or one or more other landmarks. The plurality of atlas landmarks may for example comprise, but is not limited to, a predefined number of maxillary front teeth (e.g. 3 most central teeth on both sides, i.e. teeth from a canine tooth to a right canine tooth including both canine teeth) landmarks, a predefined number of mandibular front teeth (e.g. 3 most central teeth on both sides) landmarks, a mandibular head landmark(s), a chin menton landmark, a chin notch landmark, an acanthion, i.e. nose crossing, landmark, a tragus landmark(s), an external auditory canal landmark(s), a sinus landmark(s), a TMJ landmark(s), an eye socket landmark(s) (e.g. a lower orbit of the eye socket landmark(s)), and/or a landmark of any other anatomically relevant structure. The atlas landmark data may further comprise a label, e.g. an identifier (ID), the plurality of atlas landmarks. The atlas anatomy data may typically be extracted from CT-images, e.g. CBCT-images, medical CT images, or other radiological images. Typically, these CT-images are 3D images, but the CT-images may also be 2D or 4D images. The atlas anatomy data may be extracted from a single subject or from multiple subjects. Alternatively, the atlas anatomy data may also be created without the CT-images. Thus, the atlas anatomy data may also be artificial or generic. In addition to the atlas anatomy data, the dental atlas data 1116 may further comprise atlas imaging data. The atlas imaging data may for example comprise atlas ROI data. The atlas ROI data may comprise a location of one or more possible ROI positions. For example, in case of the CT imaging, the location of the one or more possible ROI positions may comprise a location of one or more single teeth, a range of teeth, a dental arch (maxillary and/or mandibular), a TMJ(s), whole dentition and TMJs, and/or any other specific ROI position. For example, in case of the panoramic imaging, the location of the one or more possible ROI positions may comprise a location of one or more imaging layers. Preferably, the atlas ROI data may comprise the location of all possible ROI positions. The location of each possible ROI position may for example comprise a center of said possible ROI. The atlas ROI data may further comprise a label for the one or more possible ROI positions. The atlas imaging data may further comprise atlas scan trajectory data and/or any other imaging data being geometry dependent. The atlas scan trajectory data may for example comprise rotation axis data, rotation angle data, exposure parameter data, sharp layer data (in case of the panoramic imaging), and/or timing diagram data. The rotation axis data may comprise locations of a mechanical (i.e. physical) rotation axis, i.e. mechanical rotation center, of the gantry part 112 during the scan, e.g. a starting position of the scan (i.e. scan start control position), intermediate control points of the scan, and/or a scan end position (i.e. scan start control position). Alternatively or in addition, the rotation axis data may for example comprise locations of a virtual rotation axis, i.e. a virtual rotation center, of the gantry part 112 during the scan, e.g. the starting position of the scan, the intermediate control points of the scan, and/or the scan end position. The rotation angle data may for example comprise rotation angles of the gantry part 112 during the scan, e.g. scan start angle, intermediate angles, and/or scan end angle. The exposure parameter data may for example comprise information on exposure parameters during the scan trajectory and/or for each ROI position. The sharp layer data may for example comprise size, shape, and location of the sharp layer. The parts of the patient's anatomy that hit in the sharp layer are sharp in the dental X-ray image and the other parts of the patient's anatomy are blurred. The timing diagram data may for example comprise timing diagrams indicating when the emission of the beam of the X-ray is on/off for each specific ROI position. The dental atlas data 1116 may be generated from data acquired from one or more dental atlas databases. The term “dental atlas” may also be called as dental model, dental template, dental mold, dental sample, dental framework, dental artificial, dental prototype, and/or any other similar term. The dental atlas data 1116 may cover information not only from dentition but also on mandible, maxilla and/or skull or parts of them. Thus, at least the following synonyms for dental prefix in the term “dental atlas data” may exist: jaw, arch, dental arch, mandible, maxilla, and/or maxiofacial, etc. The dental atlas data 1116 may be stored into a memory part 1108 of the control system 106.

The at least one image analysis model 1118 may be formed based on previously collected reference image data. For example, the at least one image analysis model 1118 may be trained by using the previously collected reference image data. The at least one image analysis model 1118 may comprise at least one machine learning (ML)-based model and/or at least one artificial intelligent (AI)-based model. The at least one image analysis model 1118 may possibly further comprise at least one conventional image analysis model. The at least one ML-based model may be formed by applying one or more known ML techniques. According to a non-limiting example the at least one ML-based model may for example be based on ensemble of regression trees ML method and/or histogram of oriented gradients (HoG) ML method. The at least one AI-based model may be formed by applying one or more known AI techniques. According to a non-limiting example, the at least one AI-based model may for example be based on deep neural networks, deep convolutional neural networks, traditional neural networks, region-based convolutional networks, and/or region based fully convolutional networks, etc. The previously collected reference image data for the at least one image analysis model may be collected and the at least one image analysis model 1118 may be formed from the previously collected reference image data before implementation of the at least one image analysis model 1118 (e.g. in the definition of the ROI position) by the control system 106. The at least one imaging model 1118 may be stored into the memory part 1108 of the control system 106.

The previously collected reference image data may comprise image data of a large amount of people. According to a non-limiting example, the large amount of people may comprise approximately more than 1000 people. Preferably the large amount of people may comprise approximately from 2000 to 3000 people or even more. The reference image data may comprise at least one optical reference image of each person belonging to the plurality large amount of people. Preferably, the reference image data may comprise multiple optical images of each person belonging to the plurality large amount of people. The multiple optical images of each person may comprise optical images from multiple different positions of the person and/or angles of view. For example, the multiple optical images of each person may comprise at least one optical image from the front of the person, from each side of the person, a head down position, a head up position, and/or with a facial expression with a grimace (i.e. a facial expression where a dentition of the person is at least partly visible, preferably fully visible). The reference optical images of the reference image data may be still images and/or video images. The reference image data may be captured by using at least one reference imaging device 302. Preferably, the reference image data may be captured by using a plurality of reference imaging devices 302. This enables that multiple optical images may be captured substantially simultaneously from different angles of view. The at least one reference imaging device 302 may be a 2D imaging device, and/or a 3D imaging device. Preferably, the at least one reference imaging device 302 is the 3D imaging device. The description relating to the 3D imaging device and to the 2D imaging device described above referring to the at least one imaging device 104a, 104b applies also to the at least one reference imaging device 302.

According to a non-limiting example, the reference image data may be provided from one or more reference imaging locations, e.g. from one or more photo booths, 300. Each reference image location 300 may comprise a plurality of reference imaging devices 302 configured to provide at least part of the reference image data. FIG. 3 illustrates a non-limiting example of a reference imaging location, e.g. a photo booth, 300 comprising a plurality of reference imaging devices 302 configured to provide at least part of the reference image data. In the example of FIG. 3 the plurality of reference imaging devices 302 are arranged in the stereovision configuration. Thus, the reference signs 302 refers to pairs of reference imaging devices. In the example of FIG. 3 a person 304 is being photographed inside the photo booth 300 and the plurality of reference imaging devices 302 are configured to capture reference optical images of the person 304, wherein the captured reference optical images of the person 304 may be included in the reference image data.

According to an example, the at least one image analysis model 1118 may further be retrained with further reference image data collected later, e.g. during the use of the method by the dental X-ray imaging system 100. For example, the optical images 105 of the patient(s) 600 captured by the at least one optical imaging device 104a, 104b may be used as the further reference image data to further retrain the at least one image analysis mode. Alternatively or in addition, the further reference image data may be collected by using the at least one reference imaging device 302, e.g. at the one or more photo booths 300. The retrained at least one image analysis model 1118 may be stored into the memory part 1108 of the control system 106 and replace the previously stored at least one image analysis model 1118.

As discussed above the control system 106 defines the ROI position at the step 230 based on the ROI data, the at least one optical image 105, the dental atlas data 1116, and the at least one image analysis model 1118. To define the ROI position the control system 106 may first determine aligned dental atlas data of the patient 600 based on the at least one optical image 105 of the patient 600, the dental atlas data 1116, and the at least one image analysis model 1118. The aligned dental atlas data may comprise dental atlas data 1116 aligned with the patient 600, e.g. the atlas anatomy data aligned with the patient 600 and possibly also at least part of the atlas imaging data aligned with the patient 600. When the dental atlas data 1116 is aligned with the patient 600, the atlas anatomy data and possibly also at least part of the atlas imaging data may be transferred into the exact location and anatomy of the patient 600. After the determination of the aligned dental atlas data of the patient 600 the control system 106 is configured to define the ROI position based on the ROI data and the determined aligned dental atlas data of the patient 600. FIG. 4 illustrates schematically an example of a method for determining the aligned dental atlas data of the patient 600 based on the at least one optical image 105 of the patient 600, the dental atlas data 1116, and the at least one image analysis model 1118. The determined aligned dental atlas data of the patient 600 may be used in the definition of the ROI position at the step 230. Alternatively or in addition, the determined aligned dental atlas data of the patient 600 may be used in one or more other applications, e.g. in a definition of exposure parameters for the scan as will be described later in this application. To determine the aligned dental atlas data of the patient 600 at least one optical image 105 of the patient 600 where the dentition of the patient 600 is at least partly visible is needed. In other words, the at least one optical image 105 of the patient 600 used in the determining the aligned dental atlas data comprises at least one optical image where the dentition of the patient 600 is at least partly visible. Preferably at least two optical images 105 of the patient 600, where the dentition of the patient 600 is at least partly visible, captured from different angles of view may be used. This enables that the head of the patient 600 may be better covered in the optical image(s) 105.

At a step 410, the control system 106 may determine (e.g. extract) a plurality of head landmarks 502, 504, 506, 508, 510, 512 of the patient 600 based on the at least one optical image 105 of the patient 600 and the at least one image analysis model 1118. For example, the at least one image analysis model 1118 (e.g. at least one ML-based model) may be used to detect the plurality of head landmarks 502, 504, 506, 508, 510, 512 from the at least one optical image 105 of the patient 600. In other words, the at least one optical image 105 of the patient 600 may be used as the input data of the at least one image analysis model 1118 and the plurality of head landmarks 502, 504, 506, 508, 510, 512 of the patient 600 may be obtained as the output data of the at least one image analysis model 1118. The determined plurality of head landmarks 502, 504, 506, 508, 510, 512 may be presented as 3D coordinates. The determined plurality of head landmarks 502, 504, 506, 508, 510, 512 may for example comprise, but is not limited to, a predefined number of maxillary front teeth (e.g. 3 most central teeth on both sides, i.e. teeth from a canine tooth to a right canine tooth including both canine teeth) landmarks 502, a predefined number of mandibular front teeth (e.g. 3 most central teeth on both sides) landmarks 512, mandibular head landmarks 504, a chin menton landmark 506, a chin notch landmark 508, and/or an acanthion, i.e. nose crossing, landmark 510. Another non-limiting examples of the head landmarks may comprise, but is not limited to, a tragus landmark(s), an external auditory canal landmark(s), a sinus landmark(s), a TMJ landmark(s), eye socket landmark(s) (e.g. a lower orbit of the eye socket landmark(s)), and/or a landmark of any other anatomically relevant structure. The predetermined number of maxillary front teeth landmarks 502 are directly located on the maxilla bone. Similarly, the predetermined number of mandibular front teeth landmarks 512 are directly located on the mandible bone. The other landmarks of the patient 600 (e.g. the mandibular head landmarks 504, the chin menton landmark 506, the chin notch landmark 508, and/or the acanthion landmark 510) may be detected on a skin surface and the landmarks may be projected on the bone by projecting them through an average skin-subcutaneous fat thickness, i.e. by moving them inwards the amount of average skin-subcutaneous fat thickness. Alternatively or in addition, there is no need to determine both the maxillary front teeth landmarks 502 and the mandibular front teeth landmarks 512. For example, if the maxillary front teeth landmarks 502 are determined, the mandibular front teeth landmarks 512 may be determined by projecting the maxillary front teeth landmarks 502 downward a known tooth length amount on the bone.

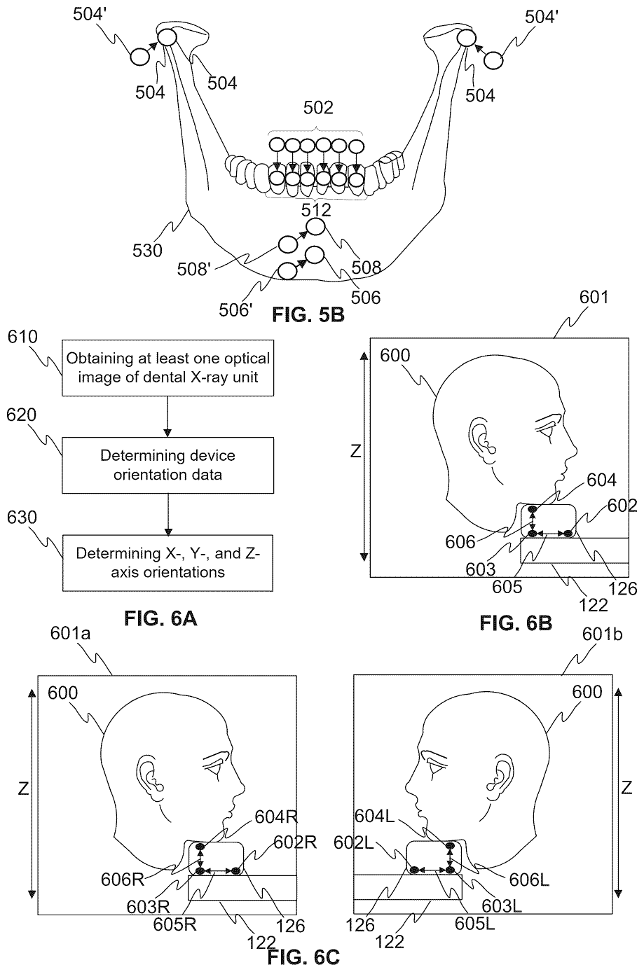

FIGS. 5A and 5B illustrate schematically a non-limiting example of the plurality of head landmarks 502, 504, 506, 508, 510, 512 of the patient 600 determined based on the at least one optical image 105 of the patient 600 and the at least one image analysis model 1118. In this example, the plurality of head landmarks 502, 504, 506, 508, 510, 512 of the patient 600 comprise three maxillary front teeth landmarks on both sides 502, the mandibular head landmarks 504, the chin menton landmark 506, the chin notch landmark 508, and the acanthion landmark 510. FIG. 5A illustrates a non-limiting example maxillary bone model 520 on which the maxillary landmarks of the determined head landmarks 502, 504, 506, 508, 510, 512 of the patient 600 are projected. In this example the maxillary landmarks comprise the three maxillary front teeth landmarks 502 on both sides and the acanthion landmark 510. The projection of the acanthion landmark 510 on the bone is illustrated with the arrow in FIG. 5A and the acanthion landmark detected on the skin surface is illustrated with the reference sign 510′. FIG. 5B illustrates a non-limiting example mandibular bone model 530 on which the mandibular landmarks of the determined plurality of head landmarks 504, 506, 508, 510, 512 of the patient 600 are projected. In this example the mandibular landmarks comprise three mandibular front teeth landmarks 512 on both sides, the mandibular head landmarks 504, the chin menton landmark 506, and the chin notch landmark 508. The projection of the mandibular head landmarks 504, the chin menton landmark 506, and the chin notch landmark 508 on the bone are illustrated with the arrows in FIG. 5B and the mandibular head landmarks, the chin menton landmark, and the chin notch landmark detected on the skin surface is illustrated with the reference signs 504′, 506′, and 508′ respectively. Also, the projection of the maxillary front teeth landmarks 502 on the bone to determine the mandibular front teeth landmarks 512 are illustrated with the arrows in FIG. 5B.

According to an example, the control system 106 may further estimate the confidence of the plurality of landmarks of the patient 600 extracted at the step 410 and/or extracted at any other step that will be described later in this application. The estimation of the confidence of the plurality of landmarks of the patient 600 is illustrated as an option step 412 in the example of FIG. 4. The confidence estimation may for example be binary and/or weighted. In the binary confidence estimation, the plurality of landmarks may be divided into two groups: 1) valid landmarks (to be used in a registration at a step 430), and 2) invalid landmarks (not used in the registration at the step 430). The registration at the step 430 will be described later in this application. In the weighted confidence estimation, each landmark gets a weight reflecting the estimated confidence. High confidence results a large weight and low confidence results a small weight. The weighted confidence estimation may assume that registration process at the step 430 may handle the weights. Alternatively, a binary labelling and removing invalid landmarks may be used. Typically, the at least one image analysis model 1118 may output all landmarks even in case of occlusion (e.g. a lip is occluding the visibility to a specific tooth, e.g. a canine tooth) and/or in case of target structure is missing (e.g. the patient 600 does not have the specific tooth, e.g. the canine tooth), and/or in case of the landmark is displaced (e.g. detected canine landmark corresponds to location of a premolar tooth). These landmarks are invalid and should not be used in the registration process at the step 430. Similarly, in the weighted confidence estimation zero weight or at least very small weight should be assigned for these invalid landmarks. The control system 106 may for example use a spatial analysis for the confidence estimation. The relative distances (distance that considers the scaling differences between different heads) between any two landmark points is roughly known in advance. Similarly, the relative angle between two lines is roughly known in advance. For example, a line connecting landmarks related to the tragus and the lower orbit of the eye socket and a line connecting landmarks related to the tragus and the acanthion 510 are known to have relative angle close to 10 degrees. A large deviation from the expected relative distance and/or relative angle indicates low confidence. Alternatively or in addition, the control system 106 may for example use a regional content analysis for the confidence estimation. In the regional content analysis, the control system 106 analyzes the content of the at least one optical image 105 close to the detected landmark. The regional content analysis may for example be a texture regional analysis, a color texture regional analysis, and/or any other regional analysis. The regional analysis may also be based Al-model trained to perform the landmark confidence estimation. Alternatively or in addition, if the at least one imaging device 104a, 104b captures optical images at different time points, the control system 106 may use temporal analysis for the confidence estimation. If the patient 600 is supported by patient support parts 124, 126, the target anatomical structures are assumed to have substantially stable spatial location over a short time interval. If the location of the landmark varies greatly over a short time interval, a low confidence may be assumed. Similarly, if the landmark remains stable, a high confidence may be assumed.

According to another example, the control system 106 may, alternatively or in addition, combine landmarks of the patient 600 of the same anatomical location and/or combine spatial information, i.e. combine landmarks detected from optical images obtained from more than one imaging device 104a, 104b. The combining of the landmarks of the patient 600 is illustrated as an option step 414 in the example of FIG. 4. According to an example, there may be multiple detected landmarks for the same anatomical location viewed from different imaging devices, if multiple imaging devices 104a, 104b are used, and/or there might be multiple landmark detections taken slightly different time (multiple temporal locations). As a result, there may be multiple landmarks to the same anatomical locations. There may be many strategies for combining multiple instances of the same landmarks into a single landmark and any of them may be used here. For example, weighted averaging schemes may be used. Some non-limiting exemplary averaging schemes may for example include a fixed weighting (e.g. arithmetic mean), a weighting according to time (e.g. more recent time points have larger weight than older samples, i.e. a temporal averaging), and/or a weighting according to the confidence, etc. This landmark combination is only an optional process, and the need of the combination may depend on the registration process at the step 430. For example, if the registration process is capable of handling unequal size point sets (e.g. an Iterative closest point (ICP) algorithm) then more than one landmark per single anatomical location may be maintained. According to another example, if more than one imaging device 104b is used, some landmarks may be visible and detected in the optical image from one imaging device 104a, 104b and other landmarks may be visible and detected in another optical image of another imaging device 104a, 104b. The detected landmarks may be presented in an internal imaging device coordinate system, which is unique for each imaging device 104a, 104b. Combining the spatial information means transforming the landmarks from different imaging device coordinate systems into one base coordinate system. One possibility is to assign one imaging device 104a, 104b as a master imaging device, where the landmarks detected in the optical images of the other (i.e. slave) imaging device(s) 104a, 104b may be transferred. The transformation between imaging devices 104a, 104b may be known from a joint calibration of the imaging devices 104a, 104b. Another option may be to agree some other fixed coordinate system, e.g. a coordinate system of the dental X-ray imaging unit 102 as the base coordinate system and transform the landmarks determined from the optical image of each imaging device 104a, 104b into the coordinate system of the dental X-ray imaging unit 102. The transformation from the imaging device coordinates into the coordinates of the dental X-ray imaging unit 102 may be known based on calibration data formed by performing a calibration of the imaging device(s) 104a, 104b to the dental X-ray imaging unit 102. The calibration may for example be performed during the dental X-ray imaging unit 102 setup. The calibration may further be repeated after any period e.g. during annual maintenance. In the calibration a calibration target comprising at least three calibration marks being visible in optical images and X-ray images may be used.

According to an example, if the control system 106 is not able to determine one or more of the plurality of head landmarks 504, 506, 508, 510, 512 of the patient 600 based on the at least one optical image 105 of the patient 600 and the at least one image analysis model 1118, the control system 106 may generate an indication to the operator of the dental X-ray imaging unit 102, via one or more user interface devices, e.g. via the user interface device 140a. In response to generating the indication for the operator, the operator may manually input the one or more missing head landmarks of the patient 600 (e.g. the one or more of the plurality of head landmarks 502, 504, 506, 508, 510 of the patient 600, which the 106 is not able to determine) via the one or more user interface devices, e.g. via the user interface device 140a. The minimum amount of the landmarks of the patient 600 to be used in the registration at the step 430 may for example be three. Preferably, the landmarks of the patient 600 to be used in the registration at the step 430 may be sampled from different parts of the patient 600 and not locating all in the same region. For example, three landmarks locating all at front teeth of the patient 600 may not be sufficient to provide accurate registration across the whole modeled region. For example, if after the estimation of the confidence of the determined landmarks of the patient 600 at the optional step 412, less than three reliable landmarks were found and/or if there are no reliable landmarks outside the central teeth region, the control system 106 may generate an indication to the operator of the dental X-ray imaging unit 102 to manually input the one or more missing head landmarks of the patient 600 via the one or more user interface devices.

At a step 420, the control system 106 may select a plurality of atlas landmarks comprised in the dental atlas data 1116. For example, I plurality of selected atlas landmarks may correspond to the plurality of head landmarks 502, 504, 506, 508, 510, 512 of the patient 600 determined at the step 410 discussed above. In other words, the number of the plurality of head landmarks 502, 504, 506, 508, 510, 512 of the patient 600 and the number of the atlas landmarks is the same. This enables a point-based pair-wise registration at the step 430. In the paired registration for each head landmark 502, 504, 506, 508, 510, 512 of the patient 600 there is exactly one atlas landmark corresponding to the same anatomical location. As discussed above, at least some the detected plurality of head landmarks 502, 504, 506, 508, 510, 512 of the patient 600 may be labelled as invalid landmarks and removed. In that case, the control system 106 may select only among the valid landmarks their corresponding counterparts from the dental atlas data 1116. In FIG. 4 the step 410 is presented before the step 420, but the step 420 may also be performed before or simultaneously with the step 410. In other words, the control system 106 may first select the plurality of atlas landmarks comprised in the dental atlas data 1116. Then the control system 106 may determine the plurality of head landmarks 502, 504, 506, 508, 510, 512 of the patient 600, wherein the plurality of head landmarks of the patient 600 correspond to the selected plurality of atlas landmarks.

At a step 430, the control system 106 may register (i.e. align) the plurality of atlas landmarks and the plurality of head landmarks 502, 504, 506, 508, 510, 512 of the patient 600 to determine the aligned dental atlas data of the patient 600, i.e. the atlas anatomy data of the dental atlas data 1116 is aligned with the patient 600. The control system 106 may for example perform the point-based pair-wise registration transform between the plurality of head landmarks 502, 504, 506, 508, 510, 512 of the patient 600 and the plurality of corresponding atlas landmarks. Alternatively, the point-based registration transform may performed be without pairing. The aligned dental atlas data of the patient 600 may for example comprise a patient specific anatomy model representing an estimation of the anatomy of the patient 600. As already discussed above the aligned dental atlas data may comprise at least the atlas anatomy data aligned with the patient 600. For example, the aligned dental atlas data may comprise estimated locations of anatomical structures of the patient 600, e.g. one or more single teeth, a range of teeth, the dental arch (maxillary and/or mandibular), the TMJ(s), the mandibular head(s), the chin menton, the chin notch, the acanthion, the tragus(es), the external auditory canal(s), the sinus(es), the eye socket(s) and/or any other anatomically relevant structure. The control system 106 may further align at the step 430 the atlas imaging data with the patient 600. Thus, the aligned dental atlas data of the patient 600 may further comprise the atlas imaging data aligned with the patient 600. For example, the control system 106 may further define one or more possible ROI positions in the anatomy model of the patient 600 based on the atlas ROI data comprised in the dental atlas data 116. Thus, the determined aligned dental atlas data may further comprise the one or more possible ROI positions in the anatomy model of the patient 600.. Alternatively or in addition, in case of the panoramic imaging, at the step 430 the control system 106 may further define initial scan trajectory data based on the atlas scan trajectory data comprised in the dental atlas data 1116. Thus, the determined aligned dental atlas data of the patient 600 may further comprise the defined initial scan trajectory data.

The dental atlas data 1116 and the plurality of head landmarks 502, 504, 506, 508, 510, 512 of the patient 600 may be presented in different coordinate systems. For example, the plurality of atlas landmarks, the atlas ROI data, and/or the atlas scan trajectory data comprised in the dental atlas data 1116 may be presented in an atlas coordinate system. The plurality of head landmarks of the patient 600 may for example be presented in the imaging device coordinate system, e.g. the imaging device coordinate system of a single imaging device, the base coordinate system (e.g. the master imaging device coordinate system or the coordinate system of the dental X-ray imaging unit 102 as discussed above), or any other known coordinate system. The atlas coordinates and the imaging device coordinates may be 2D, 3D, and/or 4D coordinates. Preferably, the atlas coordinates and the imaging device coordinates are 3D coordinates. In other words, at the step 430, the control system 106 may register the dental atlas data 1116 presented in the atlas coordinate system into the imaging device coordinate system by using the registration transform. The registration transform may be any type of transform, for example, but not limited to, a rigid transform, a rigid transform with an isotropic scaling, a rigid transform with an anisotropic scaling, an affine transform, a perspective transform, or any other linear transform, or any nonlinear or deformable transform. In addition, a regularization term may be used, if needed with some transforms in order to guide the transform to be constrained physically more likely solutions. Analytical solutions do exist in certain special cases (e.g. paired point-based registration with certain transformations). Otherwise, an iterative minimization may be used. In the iterative minimization, any minimization algorithm may be used. It may be defined that the registration is a minimization problem, where a cost function is minimized. For example, in cases, where the registration problem would be formulated as a maximization of similarity, the similarity value may be inverted (with a minus sign) to formulate the minimization problem. Preferably, a transformation that minimizes a residual registration error may be used. After the registration the residual registration error (e.g. final distances between registration points) may be known. The residual registration error indicates the accuracy of the registration. For accurate registrations small residual error may be expected, while large total residual error or a large individual error for a single point may typically indicate an inaccurate registration result. Thus, the accuracy of the registration may be evaluated based on the residual registration error. According to an example, if the residual registration error exceeds a threshold value, the registration may be assumed to be too inaccurate and the control system 106 may generate an assistance request to the operator of the dental X-ray imaging unit 102, via one or more user interface devices, e.g. via the user interface device 140a, for manually input one or more landmarks of the patient 600. For example, the control system 106 may define the one or more possible ROI positions in the anatomy model comprised in the aligned dental atlas data by using the registration transform at the step 430, e.g. the control system 106 may transform the one or more possible ROI positions indicated in the atlas ROI data into the imaging device coordinates to define the one or more possible ROI positions in the anatomy model comprised in the aligned dental atlas data. After transforming the atlas ROI data, i.e. the one or more possible ROI positions, into the imaging device coordinates, the one or more possible ROI positions are known in the imaging device coordinates. Thus, also the one or more possible ROI positions in the anatomy model comprised in the aligned dental atlas data are known. For example, in case of the CT imaging, the control system 106 may transform the one or more possible FOV positions indicated in the atlas ROI data into the imaging device coordinates to define the one or more possible FOV positions in the anatomy model of the patient 600 comprised in the aligned dental atlas data. Alternatively or in addition, in case of the panoramic imaging, the control system 106 may further define the initial scan trajectory data based on the atlas scan trajectory data by using the registration transform at the step 430, e.g. the control system 106 may transform at least one scan trajectory indicated in the atlas scan trajectory data comprised in the dental atlas data 1116 into the imaging device coordinates to define the initial scan trajectory data comprised in the aligned dental atlas data.

As discussed above after determining the aligned dental atlas data of the patient 600 the control system 106 may define the ROI position based on the ROI data and the determined aligned dental atlas data of the patient 600 at the step 230. The ROI data comprised in the scan request may indicate the target ROI. The target ROI corresponds to one possible ROI position in the aligned dental atlas data comprised in the aligned dental atlas data. Thus, the control system 106 may define that the ROI position is said one possible ROI position corresponding to the target ROI. For example, if the target ROI is a single tooth, the control system 106 may define that the ROI position is the position of said single tooth in the anatomy model comprised in the aligned dental atlas data. As the ROI position is defined in the dental arch model comprised in the aligned dental atlas data, the ROI position is presented in the imaging device coordinates. For example, in case of the CT imaging, the control system 106 may define that the FOV position is a FOV position in the anatomy model comprised in the aligned dental atlas data corresponding to the target FOV indicated in the ROI data. In case of the CT imaging, the definition of the ROI position may further comprise defining a center point of the defined ROI position, i.e. a ROI center, e.g. a FOV center. According to a non-limiting example, if the target ROI comprises a single structure, e.g. a single tooth, the control system 106 may define that the ROI center is the center of the single structure. According to another non-limiting example, if the target ROI comprises multiple structures, e.g. a range of teeth, the control system 106 may define that the ROI center is the center of the multiple structures. Alternatively or in addition, in case of the CT imaging, the control system 106 may further be configured to adjust a size of the FOV based on the determined aligned dental atlas data of the patient 600. In the CT imaging, the FOV may preferably have cylindrical shape. Thus, the adjustment of the size of the FOV based on the determined aligned dental atlas data of the patient 600 may comprise adjusting the height and/or the radius of the cylindrical FOV. As the determined aligned dental atlas data of the patient 600 enables a reliable estimation of the anatomy of the patient 600 and determination of the FOV position, the size of the FOV may be decreased so that the target FOV indicated in the ROI data is still inside the decreased FOV. Smaller FOV enables a smaller radiation dose for the patient 600 and a smaller amount of the acquired X-ray image data, which in turn leads to a faster processing of the acquired X-ray image data. For example, in case of the panoramic imaging, the control system 106 may define the position of the imaging layer so that it corresponds to the anatomic structures of the patient 600 based on the ROI data and the determined aligned dental atlas data of the patient 600.

The use of the dental atlas data 1116 with the at least one optical image 105 and the at least one image analysis model 1118 enables determination of one or more dental structures of the patient 600 that are located inside the head of the patient 600, e.g. the aligned dental atlas data of the patient 600 discussed above. The use of at least one optical image 105 of the patient 600 and the at least one image analysis model 1118 (without the dental atlas data 1116) enable determination of visible patient related data comprising one or more visible structures of the patient 600. For example, the control system 106 may further determine the visible patient related data based on the at least one optical image 105 of the patient 600 and the at least one image analysis model 1118. The visible patient related data may for example comprise head size data of the patient 600, classification data of the patient 600, height data of the patient 600, and/or width data of the patient 600. Examples of the use of the visible patient related data will be discussed later in this application.

After defining the ROI position at the step 230 as described above, the control system 106 may further define at a step 240 a scan trajectory of the parts of the dental X-ray imaging unit 102 based on the defined ROI position, the imaging mode data, and patient positioning data. According to an example, the defined ROI position may also comprise the patient positioning data. As the ROI position is defined at the step 230 by using the aligned dental atlas it enables that the defined ROI position is substantially accurate for example in comparison to an estimation of a ROI position that is typically used in the definition of the scan trajectory. The defining the scan trajectory may comprise defining a starting position of the scan trajectory (i.e. the starting position for the parts of the X-ray imaging unit 102 for the scan) and/or a motion path of the scan trajectory. The patient positioning data may comprise a position of the patient positioning, e.g. a position from which the patient 600 is supported. The patient positioning data may for example be defined based on patient support parts 124, 126 of the dental X-ray imaging unit 102. According to a non-limiting example, the position of the patient positioning may be defined based on a bite block, e.g. a bite stick, arranged for example to the chin support part 124. Alternatively, the patient positioning data may for example be defined based on optical image data collected by the at least one internal optical imaging device 104b of the dental X-ray imaging unit 102. The patient positioning data enables that the control system 106 knows the position of the head of the patient 600, e.g. the position from which the patient 600 is supported.

The motion path of the scan trajectory may depend on the imaging mode indicated in the imaging mode data. In case of the CT imaging, the motion path of the scan trajectory may for example be a circular path or a non-circular path, e.g. an elliptic path, around the rotation axis. The circular path or the non-circular path may be full rotation or partial rotation around the rotation axis. The rotation axis may be a mechanical rotation axis of the gantry part 112 or a virtual rotation axis. The mechanical rotation axis of the gantry part 112, may be oriented, i.e. aligned, with the defined ROI position as will be discussed. The virtual rotation axis may be obtained, for example, by moving the mechanical rotation axis of the gantry part 112 along a circular path, whereupon the virtual rotation axis may be formed in the center of said circular path. The non-circular rotation path may be produced, for example, by moving the gantry part 112 along a motion path deviating from the circular path, for example an elliptic path. Other techniques or alignments for the rotation axis may also be used as will be recognized by a person or ordinary skill in the art. In case of the panoramic imaging the scan trajectory may for example be substantially an arched shaped path.

When knowing the position of the head of the patient 600 based on the patient position data, the starting position of the scan trajectory may be defined based on the defined ROI position. For example, in case of a symmetrical CT-imaging, where the motion path of the scan trajectory is the circular path, the starting position of the scan trajectory may be the ROI center defined at the step 230. According to another example, in case of an off-set CT-imaging, where the motion path of the scan trajectory is the circular path, the starting position of the scan trajectory may be off-set from the ROI center defined at the step 230 by a known amount in a known direction, i.e. by a known off-set vector. As discussed above, in case of the panoramic imaging, the determined aligned dental atlas data of the patient 600 may comprise the initial scan trajectory data. In case of the panoramic imaging, the scan trajectory may be defined based on the initial scan trajectory data. According to an example, a patient specific scan trajectory may be defined based on the initial scan trajectory data. The patient specific scan trajectory may comprise the starting position of the patient specific scan trajectory and the motion path of the patient specific scan trajectory. In some cases, it may be possible that the patient specific scan trajectory may violate some basic conditions, e.g. magnification factor is not constant across the whole dental arch, because the registration transform may not be rigid. Thus, the defined patient specific scan trajectory may be adjusted (i.e. changed) to ensure that the basic conditions are not violated. The patient specific scan trajectory enables that substantially the whole dental arch of the patient 600 hits in the sharp layer. According to an example, a predefined scan trajectory may be selected based on the initial scan trajectory data. The initial scan trajectory data may comprise a plurality of predefined scan trajectories, each predefined scan trajectory comprising the starting position of said predefined scan trajectory and the motion path of said predefined scan trajectory. The predefined scan trajectory that fits best to the anatomy of the patient 600 may be selected from among the plurality of predefined scan trajectories comprise in the initial scan trajectory data, e.g. based on smallest residual registration error in the registration transform. The selected predefined scan trajectory is a good approximation for the patient 600 although not the perfect fit. Thus, even though the starting position of the predefined scan trajectory is known, this may not be the optimal starting position. If an optimal fit is not possible across the whole scan trajectory, it may be preferable to adjust the starting position the predefined scan trajectory so that an optimal match may be made near the front teeth region as the sharp layer is narrowest at that region. In other words, the starting position the predefined scan trajectory may preferably be adjusted so that at least the front teeth region hits in the sharp layer. The advantages of the use of the selected predefined scan trajectory may comprise at least that the scan trajectories and the timing diagrams etc. are pre-known and there is no technical nor mechanical limitations to apply them.

The ROI position may be presented in different coordinates than device coordinates (i.e. in the coordinates of the dental X-ray imaging unit 102). Therefore, the control system 106 may define coordinate transformation data to determine the defined ROI position in the device coordinates and thus also the starting position of the scan trajectory in the device coordinates. The coordinate transformation data may represent transformation between the coordinates in which the ROI position is presented in the device coordinates. For example, as discussed above, the ROI position defined at the step 230 may be presented in the imaging device coordinates. The control system 106 may define coordinate transformation data representing transformation between the imaging device coordinates and the device coordinates. The coordinate transformation data may for example comprise device axis orientation data in the imaging device coordinates. The device axis orientation data in the imaging device coordinates may for example comprise a Z-axis (e.g. an inferior-superior (IS) axis) orientation, a Y-axis (e.g. a posterior-anterior (PA) axis) orientation, and a X-axis (e.g. a left-right (LR) axis) orientation of the dental X-ray imaging unit 102 in the imaging device coordinates. The coordinate transformation data may be defined device orientation encoding and/or device location encoding.

FIG. 6A illustrates an example of a method for defining the coordinate transformation data by using the device orientation encoding. The method may be performed by the dental X-ray imaging system 100 discussed above. At a step 610, the control system 106 may obtain at least one optical image of the dental X-ray imaging unit 102. The at least one optical image of the dental X-ray imaging unit 102 may for example be captured by at least one internal imaging device 104b of the dental X-ray imaging unit 102. The device orientation encoding may for example be used, if the at least one internal imaging device 104b, from which the at least one optical image of the of the dental X-ray imaging unit 102 is obtained, is arranged in the gantry part 112 of the dental X-ray imaging unit 102. Preferably, the control system 106 may obtain at least two optical images of the dental X-ray imaging unit 102 at the step 610. The at least two optical images may be captured by at least two internal imaging devices 104b of the dental X-ray imaging unit 102. The control system 106 may for example obtain the at least one optical image of the dental X-ray imaging unit 102 from at least one internal imaging device 104b of the dental X-ray imaging unit 102. Alternatively, the control system 106 may obtain the at least one optical image of dental X-ray imaging unit 102 from a database into which the at least one optical image of dental X-ray imaging unit 102 captured by using the at least one internal optical imaging device 104b may be stored.

At a step 620, the control system 106 may determine (i.e. extract) device orientation data by detecting from the at least one optical image of the dental X-ray imaging unit 102 at least one device encoding mark arranged to dental X-ray imaging unit 102. The device orientation data may be encoded in the at least one device encoding mark. The control system 106 may extract the device orientation data encoded in the detected at least one encoding mark. The device orientation data may for example be encoded in the at least one device encoding mark by any geometric primitive, object, or pattern that is formed for example, but not limited to, by circles, dots, crossing, lines, rectangles, and/or triangles. The at least one device encoding mark may have a dual purpose or it may be hidden. For example, a checkerboard, a QR-code, a company name and/or logo, a brand name and/or logo, a device name and/or logo may also be used as the device encoding mark. The at least one device encoding mark may be 2D encoding mark that locates on 2D plane or 2D surface. With one 2D encoding mark two axes of the three axes of the dental X-ray imaging unit 102 may be explicitly encoded from and the third axis encoding is implicit, e.g. defined as a cross product of the two explicitly encoded axes. For explicit encoding with the 2D encoding at least two 2D encoding marks, which are not located in the same plane, are needed. Alternatively, the at least one device encoding mark may be a 3D encoding mark. With the one 3D encoding mark all three axes of the dental X-ray imaging unit 102 may be explicitly encoded from. If only one optical image of the dental X-ray imaging unit 102 is obtained the device encoding mark needs to be the 3D device encoding mark. This enables that the orientation of all three axis of the dental X-ray imaging unit 102 may be defined by using only one optical image of the dental X-ray imaging unit 102. Alternatively, the device orientation data may be determined from two differently oriented 2D device encoding marks. Also, this enables that the orientation of all three axis of the dental X-ray imaging unit 102 may be defined by using only one optical image of the dental X-ray imaging unit 102.

At a step 630, the control system 106 may determine the coordinate transformation data, i.e. the X-axis orientation, the Y-axis orientation, and the Z-axis orientation of the dental X-ray imaging unit 102, based on the device orientation data determined at the step 620. According to an example, the Z-axis orientation may be explicitly encoded in the at least one device encoding mark. According to another example, the at least one internal imaging device 104b, from which the at least one optical image data of the dental X-ray imaging unit is obtained, may be fixed to the dental X-ray imaging unit 102 so that that one side of an imaging sensor of said internal imaging device 104b is fully vertical and therefore defines the Z-axis orientation of the dental X-ray imaging unit 102 in the imaging device coordinates. According to yet another example, the X-axis orientation and the Y-axis orientation may be explicitly encoded in the at least one device encoding mark and the Z-axis orientation may then be determined implicitly as a cross product of the X-axis orientation and the Y-axis orientation. Alternatively, the Z-axis orientation and the X-axis orientation may be explicitly encoded in the at least one device encoding mark and the Y-axis orientation may then be determined implicitly as a cross product of the Z-axis orientation and the X-axis orientation. Alternatively, the Z-axis orientation and the Y-axis orientation may be explicitly encoded in the at least one device encoding mark and the X-axis orientation may then be determined implicitly as a cross product of the Z-axis orientation and the Y-axis orientation.

FIG. 6B illustrates schematically a non-limiting example of one optical image 601 of the dental X-ray imaging unit 102 for extracting the device orientation data and for determining the coordinate transformation data of the dental X-ray imaging unit 102 by using the device orientation encoding. In the example of FIG. 6B, the internal imaging device 104b from which the optical image 601 of the dental X-ray imaging unit 102 is obtained is arranged to the dental X-ray imaging device 102 on the right side of the patient 600, but alternatively the internal imaging device 104b, from which the optical image 601 of the dental X-ray imaging unit 102 is obtained, may also be arranged to the dental X-ray imaging device 102 on the left side of the patient 600. In the example of FIG. 6B one device encoding mark is arranged to dental X-ray imaging unit 102 on the same side as the internal imaging device 104b from which the optical image 601 of the dental X-ray imaging unit 102 is obtained. The device encoding mark comprises three dots 602, 603, 604 arranged into the chin support part 126 of the lower shelf 122 of the dental X-ray imaging unit 102. In the example of FIG. 6B, the X-axis and the Y-axis are explicitly encoded into the device encoding mark. However, this is only one non-limiting example for the arranging the device encoding mark to the dental X-ray imaging unit 102, and the at least one device encoding mark may be arranged also to any other part of the dental X-ray imaging unit 102 that is rigidly attached to the upper shelf 110 of the dental X-ray imaging unit 102. The upper shelf 110 determines the device axis orientation. For example, when the upper shelf 110 is not pivoting, then the at least one device encoding mark may be arranged to lower shelf 112 of the dental X-ray unit 102 (e.g. to the chin support part 126 as illustrated in the example of FIG. 6B) to the supporting column 103, or to the upper shelf 110. Alternatively, when the upper shelf 110 is pivoting, then the at least one device encoding mark needs to be arranged to the upper shelf 110 or any structure attached to upper shelf 110. The encoding mark comprising these dots may be detected from the optical image 601. A first unit vector 605 connecting the dots 602 and 603 of the encoding mark may define the Y-axis orientation of the dental X-ray imaging unit 102 in the imaging device coordinates. A second unit vector 606 connecting the dots 603 and 604 of the encoding mark may define the Z-axis orientation of the dental X-ray imaging unit 102 in imaging device coordinates. The X-axis orientation may for example be determined as the cross product of the Z-axis and the Y-axis.

FIG. 6C illustrates schematically a non-limiting example of two optical images 601a, 601b of the dental X-ray imaging unit 102 for extracting the device orientation data and for determining the coordinate transformation data of the dental X-ray imaging unit 102 by using the device orientation encoding. In the example of FIG. 6C, the first internal imaging device 104b, from which the optical image 106a is obtained, is arranged to the dental X-ray imaging device 102 on the right side of the patient 600 and the second internal imaging device 104b, from which the optical image 601b is obtained, is arranged to the dental X-ray imaging device 102 on the left side of the patient 600. In the example of FIG. 6C two device encoding marks are arranged to dental X-ray imaging unit 102 so that one device encoding mark is arranged on each side of the dental X-ray imaging unit 102. The device encoding mark on the right side comprises three dots 602R, 603R, 604R arranged into the chin support part 126 of the lower shelf 122 of the dental X-ray imaging unit 102. The device encoding mark on the left side comprises three dots 602L, 603L, 604L arranged into the chin support part 126 of the lower shelf 122 of the dental X-ray imaging unit 102. The encoding marks comprising these dots may be detected from the two optical images 601a, 601b. A first right unit vector 605R connecting the dots 602R and 603R of the encoding mark on the right side may define the Y-axis orientation of the dental X-ray imaging unit 102 in the imaging device coordinates. A second right unit vector 606R connecting the dots 603R and 604R of the encoding mark on the right side may define the Z-axis orientation of the dental X-ray imaging unit 102 in the imaging device coordinates. A first left unit vector 605L connecting the dots 602L and 603L of the encoding mark on the left side may define the Y-axis orientation of the dental X-ray imaging unit 102 in the imaging device coordinates. A second left unit vector 606L connecting the dots 603L and 604L of the encoding mark on the left side may define the Z-axis orientation of the dental X-ray imaging unit 102 in imaging device coordinates. The X-axis orientation may for example be determined by using combined information on both encoding marks by line connecting the same dot in the left and the right side to form line that is parallel to the X-axis. The use of two optical images for extracting the device orientation data enables providing maximum amount of data for the extraction. Furthermore, the use of two optical images for extracting the device orientation data enables explicit definition of the orientations of all three axes, i.e. X-axis, Y-axis, and Z-axis.

In the device location encoding, the control system 106 may obtain at least one optical image of the dental X-ray imaging unit 102. The at least one optical image of the dental X-ray imaging unit 102 may for example be captured by at least one internal imaging device 104b of the dental X-ray imaging unit 102. The device location encoding may for example be used, if the at least one internal imaging device 104b, from which the at least one optical image of the of the dental X-ray imaging unit 102 is obtained, is arranged into any part of the dental X-ray imaging unit 102 that is rigidly attached to the upper shelf 110 of the dental X-ray imaging unit 102. In the device location encoding, at least one device location and direction encoding mark may be arranged into the gantry part 112 of the dental X-ray imaging unit 102. The at least one device location encoding mark may encode at least one gantry point (defined here as a gantry origin) and at least a horizontal gantry axis (e.g. X-axis of the gantry part 112 or Y-axis of the gantry part 112). The description relating to the at least one device orientation encoding mark described above applies also to the at least one device location encoding mark. The vertical gantry axis (e.g. Z-axis of the gantry part 112) may be defined from the imaging device orientation or it may be encoded in the device location encoding mark. After defining the vertical axis and the horizontal axis, the other horizontal axis may be defined as a cross product of the defined vertical axis and the defined horizontal axis. The defined axes of the gantry part 112 may define a gantry specific coordinate system. A rotation center location of the gantry part 112 in the gantry coordinate system may be defined by a calibration. Any movements of the dental X-ray imaging unit 102 do not change the rotation center location of the gantry part 112 with respect the gantry coordinate system. If the at least one internal imaging device 104b, from which the at least one optical image of the dental X-ray imaging unit 102 is obtained, is arranged into any other part of the dental X-ray imaging unit 102 than the gantry part 112 and any part rigidly attached to the upper shelf 110 of the dental X-ray imaging unit 102 (e.g. in the support column 103), a combination of the device orientation encoding and the device location encoding may be used to define the coordinate transformation data. Alternatively or in addition, if the at least one optical image of the dental X-ray imaging unit 102 is obtained from at least one external imaging device 104a, the combination of the device orientation encoding and the device location encoding may be used to define the coordinate transformation data.

After defining the scan trajectory at the step 240 as discussed above, the control system 106 may further control at a step 250 the parts of the dental X-ray imaging unit 102 to scan the patient 600 according to the defined scan trajectory in order to acquire the dental X-ray image data of the patient 600. The control system 106 may define the current position of the rotation axis of the gantry part 112 based on the calibration data of the at least one imaging devices 104a, 104b to the dental X-ray imaging unit 102. To control the gantry part 112 of the dental X-ray imaging unit 102 to move from the current position of the rotation axis of the gantry part 112 into the starting position of the scan trajectory, the control system 106 may define device transformation data. The device transformation data may indicate the transform in the device coordinates needed to move the rotation axis of the gantry part 112 from the current position into the starting position. The control system 106 may be configured to control the parts of the dental X-ray imaging unit 102 to move to the starting position of the scan trajectory (i.e. the defined ROI position) based on the defined device transformation data. The movements of the parts of the dental X-ray imaging unit 102, e.g. the gantry part 112, may be linear and/or rotational. As a result, the rotation axis of the gantry part 112 is moved into the defined starting position of the scan trajectory. Next the control system 106 may control the gantry part 112 to rotate according to defined motion path of the scan trajectory around the rotation axis of the gantry part 112.

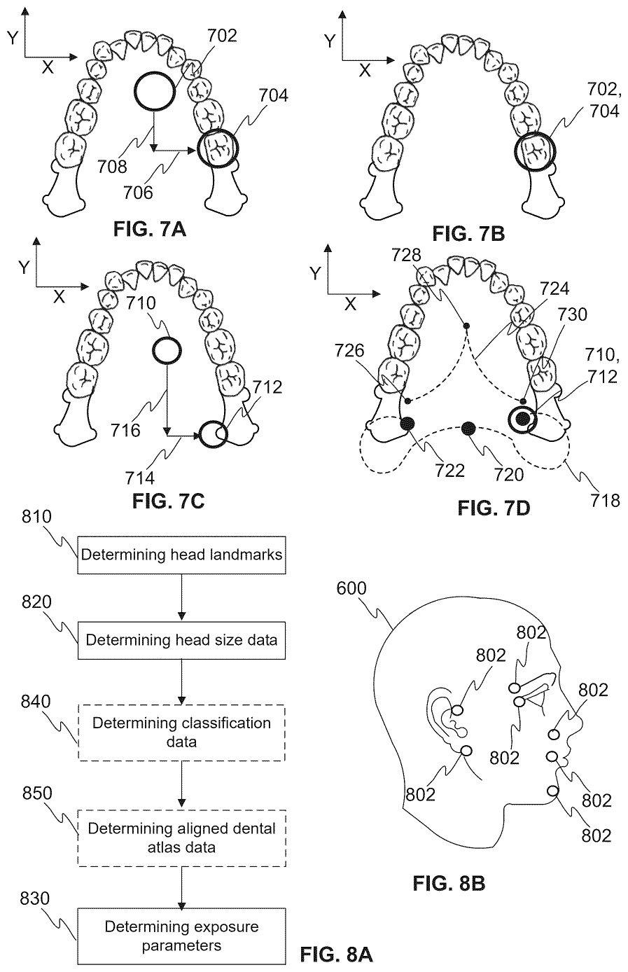

FIGS. 7A and 7B illustrate a non-limiting example of the movement of the gantry part 112 from the current position of the rotation axis of the gantry part 112 into the starting position of the scan trajectory based on the device transformation data in case of the CT-imaging. FIG. 7A illustrates a situation, where the rotation axis of the gantry part 112 is in the current position (i.e. before the controlling of the movement of the gantry part 112). The current position of the rotation axis of the gantry part 112 is illustrated with the reference sign 702 and the starting position of the scan trajectory is illustrated with the reference sign 704. In this example, the device transformation data comprises the X-directional transform illustrated with a vector 706 and the Y-directional transform illustrated with a vector 708. FIG. 7B illustrates a situation after the controlling the movement of the gantry part 112 from the current position 702 of the rotation axis of the gantry part 112 into the starting position of the scan trajectory 704. In other words, in the situation of FIG. 7B the rotation axis of the gantry part 112 is placed in the starting position of the scan trajectory 704. For example, in case of the CT imaging, where the motion path is cylindrical path, the rotation axis of the gantry part 112 is placed in the defined FOV, i.e. the rotation axis of the gantry part 112 is parallel with the center of the defined FOV. According to another example, in case of the CT imaging with the off-set scan the rotation axis of the gantry part 112 may be slightly off-set from the center of the defined FOV and the gantry part 112 moves across the FOV during the scan. Therefore, in the off-set scan, the starting position of the scan trajectory 704 is offset from the center of the defined FOV.

FIGS. 7C and 7D illustrate a non-limiting example of the movement of the gantry part 112 from the current position of the rotation axis of the gantry part 112 into the starting position of the scan trajectory based on the device transformation data in case of the panoramic imaging. FIG. 7C illustrates a situation, where the rotation axis of the gantry part 112 is in the current position (i.e. before the controlling of the movement of the gantry part 112). The current position of the rotation axis of the gantry part 112 is illustrated with the reference sign 710 and the starting position of the scan trajectory is illustrated with the reference sign 712. In this example, the device transformation data comprises the X-directional transform illustrated with a vector 714 and the Y-directional transform illustrated with a vector 716. FIG. 7D illustrates a situation after the controlling the movement of the gantry part 112 from the current position 710 of the rotation axis of the gantry part 112 into the starting position of the scan trajectory 712. In other words, in the situation of FIG. 7D the rotation axis of the gantry part 112 is placed in the starting position of the scan trajectory 712. In the example of FIG. 7D an example of the motion path of the scan trajectory for the mechanical rotation axis, i.e. the mechanical rotation center, of the gantry part 112 in the panoramic imaging is further illustrated with the reference sign 718. The motion path 718 of the scan trajectory starts from the starting position 712 of the scan trajectory. The halfway of the motion path 718 of the scan trajectory is illustrated with the point 720 and the end of the motion path 718 of the scan trajectory is illustrated with the point 720 is illustrate with the point 722. Furthermore, because the gantry part 112 rotates according to the defined motion path 718 of the scan trajectory around the mechanical rotation axis of the gantry part 112, a virtual rotation axis of the gantry part 112 is also formed. In the example of FIG. 7D, the motion path of the virtual rotation axis of the gantry part 112 is further illustrated with the reference sign 724. A starting position of the motion path 724 of the virtual rotation axis of the gantry part 112 is illustrated with the point 726. The halfway of the motion path 724 of the virtual rotation axis of the gantry part 112 is illustrated with the point 728 and the end of the motion 724 of the virtual rotation axis of the gantry part 112 is illustrate with the point 730.

According to an example, the control system 106 may alternatively or in addition determine exposure (i.e. radiation) parameters for the scan of the patient 600 based on the imaging mode data, the at least one optical image 105 of the patient, and the at least one image analysis model 1118. The exposure parameters may comprise, but is not limited to, radiation power, radiation dose, and/or radiation time, etc. To determine the exposure parameters the control system 106 may first determine the head size data of the patient 600 based on the at least one optical image 105 of the patient 600 and the at least one image analysis model 1118. Then the control system 106 may use the determined head size data of the patient 600 and the imaging mode data in the determination of the exposure parameters. FIG. 8A illustrates schematically an example of a method for determining the exposure parameters for the scan of the patient 600.

At a step 810, the control system 106 may determine a plurality of head landmarks of the patient 600 based on the at least one optical image of the patient 600 and the at least one image analysis model. The plurality of head landmarks of the patient 600 may for example correspond at least partly to the head landmarks 502, 504, 506, 508, 510 of the patient 600 as defined referring to the step 410 discussed above. Alternatively or in addition, the plurality of head landmarks of the patient 600 may comprise any other head landmarks 802 of the patient 600. FIG. 8B illustrates schematically an example of the plurality of head landmarks 802 of the patient 600 that may be determined for defining the head size data of the patient 600. In FIG. 8B only one side of the head of the patient 600 is illustrated and thus also the plurality of head landmarks 802 of the patient 600 on one side of the head of the patient 600 are illustrated, but corresponding head landmarks 802 of the patient 600 may also be determined on the other side of the head of the patient 600. For example, the at least one image analysis model 1118 (e.g. at least one ML-based model) may be used to detect the plurality of head landmarks 802 of the patient 600 from the at least one optical image 105 of the patient 600. In other words, the at least one optical image 105 of the patient 600 may be used as the input data of the at least one image analysis model 1118 and the plurality of head landmarks 802 of the patient 600 may be obtained as the output data of the at least one image analysis model 1118.

At a step 820, the control system 106 may determine the head size data of the patient 600 based on the plurality of head landmarks 802 of the patient 600 determined at the step 810. The head size data may for example comprise an estimation of the head size of the patient 600. The determination of the head size data of the patient 600 may further comprise use of anthropometric measures. With the anthropometric measures population statistics may be known e.g. head size may larger than 90 percent individuals in that population (e.g. adult male). The plurality of head landmarks 802 of the patient 600 may further include points used in the anthropometric measures (e.g. nasion, eye corners, tragus, chin menton and/or any point that may be used in the anthropometric head measures). The head size of determines how much there is tissue. The more there is tissue the higher exposure is needed.

At a step 830, the control system 106 may use the head size data of the patient 600 determined at the step 820 in the determination of the exposure parameters. For example, the head size data of the patient 600 may be used to select values for the exposure parameters. In addition to the head size the values for the exposure parameters may depend on the imaging mode. As discussed above the imaging mode is indicated in the image mode data. The exposure parameters may for example be based on tabulated values. These tabulated values may comprise parameters tabulated based on imaging mode, and/or head size. There may be a single population (i.e. only head size matters). In addition, there may be different tables for different populations by age and/or gender. In that case classification data may be used to select the correct population table. The determination of the classification data of the patient 600 will be described later in this application. In addition to the tabulated values, the exposure parameters may be determined by an equation based on the head size data and optionally the classification data. There may also be different equations for different populations and the correct equation may be selected based on the patient classification data. After the determination of the exposure parameters, the control system 106 may provide the determined exposure parameters via one or more user interface devices, e.g. the user interface device 140a, for review and approval by the operator.

According to an example, the control system 106 may further determine classification data of the patient 600 and also use the classification data in the determination of the exposure parameters at the step 830. The control system 106 may determine the classification data of the patient 600 based on the at least one optical image 105 of the patient 600 and the at least one image analysis model 118. The classification data may for example comprise an estimation of an age of the patient 600 and/or an estimation of a gender of the patient 600. In other words, the control system 106 may estimate the age of the patient 600 and/or the gender of the patient 600 from the at least one optical image 105 of the patient 600 by using at least one image analysis model 1118, e.g. at least one AI-based model. The estimation of the age of the patient 600 may for example be an estimation of an age category, e.g. a child, a teen, an adult, or an elderly. This optional step of determining the classification data of the patient 600 is illustrated in the example of FIG. 8A as an optional step 840. In FIG. 8A the step 820 is presented before the step 840, but the optional step 840 may also be performed before or simultaneously with the step 820.

According to another an example, the control system 106 may alternatively or in addition use the aligned dental atlas data of the patient 600 in the determination of the exposure parameters. The aligned dental atlas data of the patient 600 may for example be defined as discussed above referring to the method steps 410-430 of FIG. 4. This optional step of determining the aligned dental atlas data of the patient 600 is illustrated in the example of FIG. 8A as an optional step 850. In FIG. 8A the steps 820 and 830 are presented before the step 850, but the steps 820, 830, and 840 may also be performed in any other order or simultaneously.

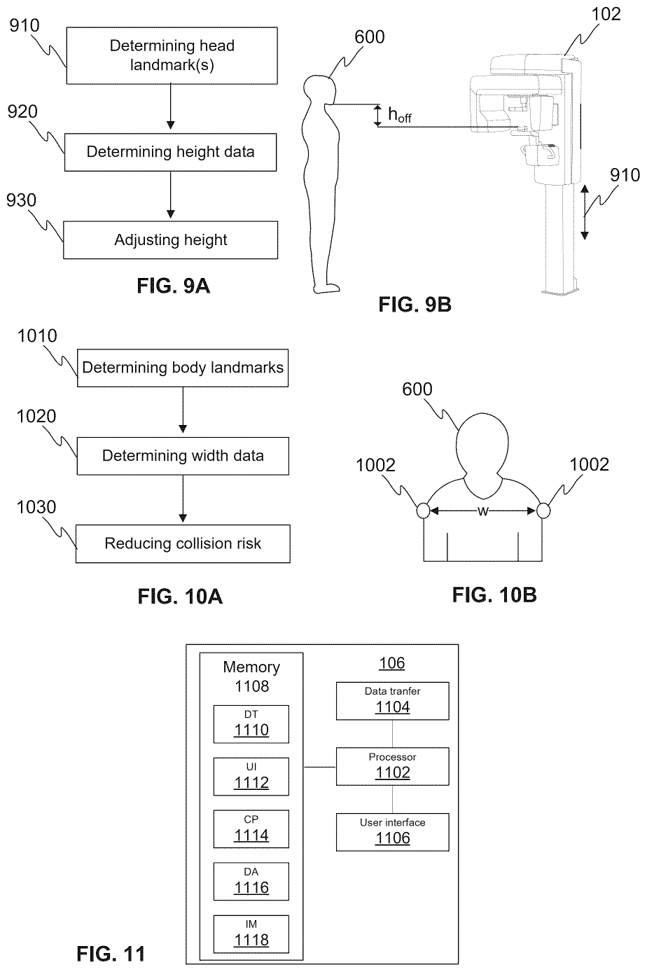

According to yet another example, the control system 106 may alternatively or in addition adjust the height of the parts of the dental X-ray imaging unit 102 based on the at least one optical image 105 of the patient 600 and the at least one image analysis model 1118. The height of the part of the dental X-ray imaging unit 102 may be adjusted by moving the carriage 101 up or down along the supporting column 103 in the height direction Z by means of the guide motor. To adjust the height of the parts of the dental X-ray imaging unit 102 the control system 106 may first determine height data of the patient 600 based on the at least one optical image 105 of the patient 600 and the at least one image analysis model 1118. Then the control system 106 may use the determined height data of the patient 600 to adjust the height of the parts of the dental X-ray imaging unit 102. FIG. 9A illustrates schematically an example of a method for adjusting the height of the parts of the dental X-ray imaging unit 102.

At a step 910, the control system 106 may determine at least one head landmark of the patient 600 based on the at least one optical image of the patient 600 and the at least one image analysis model. The at least one of head landmark of the patient 600 may be one of the head landmarks 502, 504, 506, 508, 510, 512, 802 of the patient 600 as defined referring to the step 410 and/or the step 810 discussed above. Alternatively or in addition, the at least one of head landmark of the patient 600 may be any other at least one head landmark of the patient 600. For example, the at least one head landmark of the patient 600 may be the chin menton 506 of the patient 600. For example, the at least one image analysis model 1118 (e.g. at least one ML-based model) may be used to detect the at least one head landmark of the patient 600 from the at least one optical image 105 of the patient 600. In other words, the at least one optical image 105 of the patient 600 may be used as the input data of the at least one image analysis model 1118 and the at least one head landmark of the patient 600, e.g. the chin menton 506 of the patient 600, may be obtained as the output data of the at least one image analysis model 1118.

At a step 920, the control system 106 may determine the height data of the patient 600 based on the at least one of head landmark of the patient 600 determined at the step 910. The height data may for example comprise a height-off-set value (hoff) between the at least one head landmark of the patient 600 and at least one reference height point in the dental X-ray imaging unit 102 and/or an absolute distance between the at least one head landmark of the patient 600 and a floor. The at least one reference height point in the dental X-ray imaging unit 102 may for example comprise a top of the chin support part 124. For example, the location of the optical imaging device configured to capture the at least one optical image 105 used in this example with respect to the at least one reference height point in the dental X-ray imaging unit 102 may need to be known to define the height-off-set value between the at least one head landmark of the patient 600 and the at least one reference height point in the dental X-ray imaging unit 102. FIG. 9B illustrates schematically an example illustrating an example of the height off-set value (hoff) between the at least one head landmark of the patient 600 (e.g. the chin menton 506 of the patient 600 in this example) and at least one reference height point in the dental X-ray imaging unit 102 (e.g. the top of the chin support part 124 in this example). At a step 930, the control system 106 may adjust the height of the parts of the dental X-ray imaging unit 102 based on the height data of the patient 600 determined at the step 920. The possible adjustment directions (up and down) of the height of the parts of the dental X-ray imaging unit 102 is illustrated with the arrow 910 in the example of FIG. 9B.

According to yet another example, the control system 106 may alternatively or in addition reduce a collision risk between the patient 600 and the gantry part 112 of the dental X-ray imaging unit 102 based on the at least one optical image 105 of the patient 600 and the at least one image analysis model 1118, e.g. at least one ML-based model. To reduce the collision risk, the control system 106 may first determine width data of the patient 600 based on the at least one optical image 105 of the patient 600 and the at least one image analysis model 1118. Then the control system 106 may use the determined width data of the patient 600 in the reducing of the collision risk. FIG. 10A illustrates schematically an example of a method for reducing the collision risk between the patient 600 and the gantry part 112 of the dental X-ray imaging unit 102.

At a step 1010, the control system 106 may determine a plurality of body landmarks 1002 of the patient 600 based on the at least one optical image 105 of the patient 600 and the at least one image analysis model 1118. The plurality of body landmarks 1002 may for example comprise shoulders of the patient 600. For example, the at least one image analysis model 1118 (e.g. at least one ML-based model) may be used to detect the shoulders of the patient 600 from the at least one optical image 105 of the patient 600. In other words, the at least one optical image 105 of the patient 600 may be used as the input data of the at least one image analysis model 1118 and the plurality of body landmarks 1002 of the patient 600, e.g. the shoulders of the patient 600, may be obtained as the output data of the at least one image analysis model. FIG. 10B illustrates schematically an example of the plurality of body landmarks 1002 of the patient 600 (e.g. the shoulders of the patient 600) that may be determined for defining the width data of the patient 600. Alternatively, at the step 1010, the control system 106 may determine extreme points of the body of the patient 600, e.g. shoulders of the patient 600, from the at least one optical image 105 of the patient 600.

At a step 1020, the control system 106 may determine the width data of the patient 600 based on the plurality of body landmarks 1002 of the patient 600 or the extreme points of the body of the patient 600 determined at the step 1010. The width data may for example comprise a width of the patient 600, e.g. a distance between body landmarks 1002 of the patient 600, e.g. a distance between the shoulders of the patient 600. In the example of FIG. 10B the distance (w) between the shoulders 1002 of the patient 600 is illustrated.

At a step 1030, the control system 106 use the width data of the patient determined at the step 1020 to reduce a collision risk between the patient 600 and the gantry part 112 of the dental X-ray imaging unit 102. For example, the control system 106 may use the determined width data of the patient 600 in a guidance of the patient 600 and/or the operator of the dental X-ray imaging unit 102 during an entry of the patient 600 to the dental imaging unit 102 via one or more user interface devices e.g. via a display device and/or a loudspeaker device. The one or more user interface device may comprise the user interface device 140a and/or one or more other user interface devices. For example, the guidance may comprise guidance of a suitable grip to be taken by the patient 600 to handles 128 of the dental X-ray imaging unit 102. The suitable grip may be selected by using the determined width data so that the collision risk between the patient 600 and the gantry part 112 of the dental X-ray imaging unit 102 may be reduced.

According to yet another example, the control system 106 may alternatively or in addition produce patient position correction data for the patient positioning. The patient position correction data may be produced based on the at least one optical image 105 of the patient 600 and the at least one image analysis model 1118. In this example, the at least one optical image 105 of the patient 600 may for example be captured by at least one internal imaging device 104b during the patient positioning. For example, the patient 600 may be initially positioned, e.g. by means of at least one of the patient support parts 124, 126, and then the at least one optical image 105 of the patient 600 may be captured for producing the patient position correction data. The control system 106 may then define the patient position correction data based on the at least one optical image 105 of the patient 600 and the at least one image analysis model 1118. For example, the control system 106 may determine a plurality head landmarks of the patient 600 based on the at least one optical image 105 of the patient 600 and the at least one image analysis model 1118. For example, the at least one image analysis model 1118 (e.g. at least one ML-based model) may be used to detect the plurality of head landmarks from the at least one optical image 105 of the patient 600. In other words, the at least one optical image 105 of the patient 600 may be used as the input data of the at least one image analysis model 1118 and the plurality of head landmarks of the patient 600 may be obtained as the output data of the at least one image analysis model 1118. The determined plurality of head landmarks of the patient 600 may for example comprise one or more of the head landmarks 502, 504, 506, 508, 510, 512, 802 discussed above referring to step 410 of FIG. 4 and/or referring to step 810 of FIG. 8A. Alternatively or in addition, the determined plurality of head landmarks may for example comprise one or more of other head landmarks of the patient 600. The control system 106 may determine the patient position correction data based on the determined plurality of head landmarks of the patient 600. The patient position correction data may comprise indication of one or more position errors in the patient position and/or respective correction to compensate the one or more position errors in the patient position. The one or more position errors in the patient position may comprise, but is not limited to, head nod, head twist, and/or head turn, etc., as will be described later in this application. According to an example, the control system 106 may use the determined patient position correction data in a guidance of the patient 600 and/or the operator of the dental X-ray imaging unit 102 to compensate the one or more position errors in the patient position via one or more user interface devices e.g. via a display device and/or a loudspeaker device. The one or more user interface device may comprise the user interface device 140a and/or one or more other user interface devices. Alternatively or in addition, the control system 106 may use the patient position correction data to control the parts of the dental X-ray imaging unit 102 to move according to the patient position correction data to compensate the one or more position errors in the patient position. Alternatively, the at least one internal imaging devices 104b may for example be used for the motion correction.

The correction of the head twist and/or head nod may be considered as a correction of out-of-plane position error correction. Next an example of producing the patient position correction data to compensate (i.e. correct) the head twist is described. Assuming that a Y-axis is coming from behind out of the nose of the patient 600, a rotation around this Y-axis is the head twist. The head twist may for example be detected based on two head landmarks at the same level in both sides of the face of the patient 600. For example, outer corners of the eyes as the two head landmarks may be used to determine the head twist. Alternatively, other two head landmarks, e.g. ear tragus, may also be used as the two head landmarks to determine the twist orientation. A line may be formed between the two head landmarks and an angle between the formed line and a reference horizontal line indicates the amount of head twist and the direction of the head twist. The produced patient position correction data may comprise the formed line between the two head landmarks and the formed angle between the formed line and the reference horizontal line. The head twist may for example be corrected by using the produced patient position correction data in the guidance of the patient 600 and/or the operator of the dental X-ray imaging unit 102 as discussed above. The guidance may for example be, but is not limited to, a visual guidance. The head twist may for example be corrected after obtaining the at least one optical image at the step 210 discussed above, but before using the dental atlas data at the step 230 discussed above. Next an example of producing the patient position correction data to compensate the head nod is described. Assuming that an X-axis is going through both ears of the patient 600, a rotation around this X-axis is the head nod. The head nod may for example be detected based on two head landmarks of the patient 600 which may depend on the imaging mode. In the CT-imaging the two head landmarks may for example be the nose ala and the ear tragus. In the panoramic imaging the two head landmarks may for example be the ear tragus and the eye socket (e.g. the lower orbit of the eye socket). A line may be formed between the two head landmarks and an angle between the formed line and a reference horizontal line indicates the amount of head nod and the direction of the head nod. In the CT-imaging the formed line may for example be called as a Campers line. In the panoramic imaging the formed lime may for example be called as a Frankfort horizontal line (FH-line). The produced patient position correction data may comprise the formed line between the two head landmarks and the formed angle between the formed line and the reference horizontal line. The head nod may for example be corrected by using the produced patient position correction data in the guidance of the patient 600 and/or the operator of the dental X-ray imaging unit 102 as discussed above. The guidance may for example be, but is not limited to, a visual guidance. Alternatively or in addition, the head nod may be corrected by controlling the patient support parts 124, 126 to move up (in case of nod down) or down (in case of nod up) until the formed line between the two head landmarks is fully horizontal, i.e. parallel with the reference horizontal line, by utilizing the produced patient position correction data. The head nod may for example be corrected after obtaining the at least one optical image at the step 210 discussed above and after receiving the scan request at the step 220 discussed above, but before using the dental atlas data at the step 230 discussed above. The out-of-plane position error correction enables that the dental atlas data 1116 may be used in the determination of the one or more dental structures of the patient 600 that are located inside the head of the patient 600, e.g. the aligned dental atlas data of the patient 600 discussed above.

The correction of the head turn may be considered as a correction of plane rotation. Next an example of producing the patient position correction data to compensate the head turn is described. The head turn correction may be performed after defining the ROI position at the step 230 discussed above. After the defining the ROI position the imaging plane is straight. However, the imaging plane may be rotated with respect to the dental X-ray imaging unit 102. It may be assumed that the posterior-anterior axis (PA-axis) of the patient 600 should be aligned with the posterior-anterior axis (PA-axis) of the dental X-ray imaging unit 102, which is the main axis of the upper shelf 110 of the gantry part 112. A deviation of the PA-axis of the patient 600 from the PA-axis of the dental X-ray imaging unit 102 may be caused by the patient 600 turning his/her head horizontally left or right. Assuming that there is a Z-axis coming from the feet of the patient 600 through a center of the head of the patient 600, a turn around this Z-axis causes the head turn. The head turn may for example be detected based on two head landmarks at the same level in both sides of the face of the patient 600. For example, the ear tragus as the two head landmarks may be used to determine the head turn. A line may be formed between the two head landmarks to determine an LR-axis (from left tragus to right tragus) of the patient 600. After correcting a plane-motion the IS-axis of the dental X-ray imaging unit 102 may be a good estimate for the IS-axis of the patient 600. The IS-axis and the PA-axis of the dental X-ray imaging unit 102 may be known or predefined. The PA-axis is orthogonal with respect to the LR-axis and the IS-axis, thus the PA-axis of the patient 600 may be defined as a cross product of the LR-axis and the IS-axis. An angle between the PA-axis of the patient 600 and the PA-axis of the dental X-ray imaging unit 102 indicates the amount of head turn and the direction of the head turn. The produced patient position correction data may comprise the formed angle between the PA-axis of the patient 600 and the PA-axis of the dental X-ray imaging unit 102. For example, in case of the CT-imaging, where the scan trajectory is not typically moving along the PA-axis but the scan trajectory is rather rotating around the ROI, the head turn may be corrected by controlling a start rotation angle of the gantry part 112 to be rotated in an opposite direction and an equal amount than the formed angle between the PA-axis of the patient 600 and the PA-axis of the dental X-ray imaging unit 102 comprised in the produced patient position corrections data. In case of the panoramic imaging the head turn may for example be corrected by using the produced patient position correction data in the guidance of the patient 600 and/or the operator of the dental X-ray imaging unit 102 as discussed above. The guidance may for example be, but is not limited to, a visual guidance. Alternatively or in addition, in case of the panoramic imaging the head turn may be corrected by controlling turning of the PA-axis of the dental X-ray imaging unit 102 so that it is aligned with PA-axis of the patient 600, e.g. by pivoting the upper shelf 110 according to formed angle between the PA-axis of the patient 600 and the PA-axis of the dental X-ray imaging unit 102 comprised in the produced patient position corrections data. If the pivot point of the upper shelf 110 differs from the head turn center, the pivoting may cause non-wanted movements in X-and/or Y-axis directions with respect to the head of the patient 600. These non-wanted movements in the X-and Y-axis directions may be compensated for example with equal size counter movements in X-and Y-axis directions. Alternatively or in addition, in case of the panoramic imaging the head turn may be corrected by defining the patient specific scan trajectory as discussed above, as the patient specific scan trajectory natively takes into account if the head of the patient is turned. Alternatively or in addition, in case of the panoramic imaging the head turn may be corrected by minimizing a rotation error at the region of the defined ROI position, e.g. by manipulating (i.e. optimizing) the starting position of the scan trajectory and/or the start rotation angle of the gantry part 112.

FIG. 11 illustrates a schematic example of the control system 106 of the dental X-ray imaging system 100. The control system 106 may comprise a processor part 1102, a data transfer part 1104, a user interface part 1106, and a memory part 1108. The processor part 1102 is configured to perform user and/or computer program (software) initiated instructions, and to process data. The processor part 1102 may comprise at least one processor. The memory part 1108 is configured to store and maintain data. The data may be instructions, computer programs, and any data files. The memory part 1108 may comprise at least one memory. The memory part 1108 may further comprise at least a data transfer application 1110 in order to control the data transfer part 1104, a user interface application 1112 in order to control the UI part 1106, and a computer program (code) 1114 in order to control the operations of the control system 106. The memory part 1108 and the computer program 1114, together with the processor part 1102, may cause the control system 106 at least to implement one or more method steps and/or operations of the control system 106 as described above.

The data transfer part 1104 may be configured to send control commands other units, e.g. the dental X-ray imaging unit 1102. In addition, the data transfer part 1104 may receive data from other units, e.g. the dental X-ray imaging unit 102, the at least one optical imaging device 104a, 104b, the user interface part 140b, the database(s) and/or any other external units.

The user interface (UI) part 1106 may be configured to input control commands, to receive information and/or instructions, and to display information. The UI part 1106 may comprise at least a display, a screen, a touchscreen, at least one function key, a keyboard, a wired or wireless remote controller, or any other user input and/or output device.

The computer program 1114 may be a computer program product that may be comprised in a tangible, non-volatile (non-transitory) computer-readable medium bearing the computer program code 1114 embodied therein for use with a computer, i.e. the control system 106.

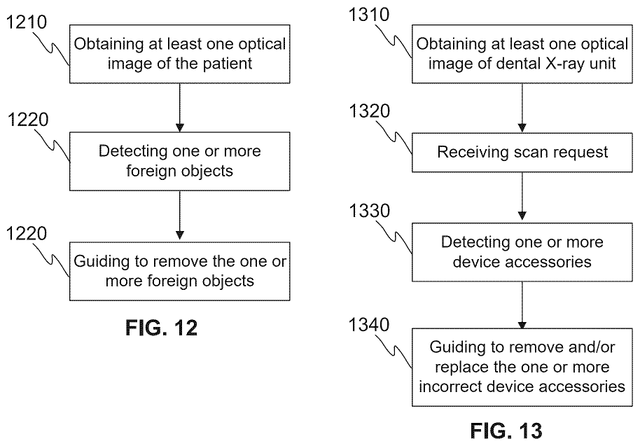

FIG. 12 illustrates schematically an example of a method for detecting a patient readiness. The method may be performed by the dental X-ray system 100 discussed above. Foreign objects in head-neck region of the patient 600 may cause artefacts in the X-ray images. Typically, the foreign objects may be accessories of the patient 600, e.g. spectacles, ear rings, nose ring, hair accessories, necklace, and/or any similar. Alternatively or in addition, the foreign object may be a wrong protective gear given by the operator of the dental X-ray imaging unit 102. For example, a thyroid collar will destroy the panoramic X-ray images. At a step 1210, the control system 106 may obtain at least one optical image 105 of the patient 600. The at least one optical image 105 of the patient 600 may be captured by using the at least one optical imaging device 104a, 104b of the dental X-ray system 102, e.g. the at least one external imaging device 104a and/or the at least one internal imaging device 104b, for example similarly as at the step 210 discussed above. At a step 1220, the control system 106 may detect one or more foreign objects based on the obtained at least one optical image 105 of the patient 600 and the at least one image analysis model 1118. In other words, the control system 106 may detect the one or more foreign objects from the at least one optical image 105 of the patient 600 by using at least one image analysis model 1118, e.g. at least one AI-based model. The detected one or more foreign objects may further be classified by using the at least one image analysis model 1118. At a step 1230, in response to the detection of the one or more foreign objects at the step 1220, the control system 106 may guide via one or more user interface devices e.g. via a display device and/or a loudspeaker device, the patient 600 and/or the operator of the dental X-ray imaging unit 102 to remove the detected one or more foreign objects. The one or more user interface device may comprise the user interface device 140a and/or one or more other user interface devices.

FIG. 13 illustrates schematically an example of a method for detecting a device readiness. The method may be performed by the dental X-ray system 100 discussed above. Different device accessories may be used in the dental X-ray imaging with the dental X-ray imaging unit 102. The required device accessories may depend on the imaging mode. At a step 1310, the control system 106 may obtain at least one optical image of the dental X-ray imaging unit 102. The at least one optical image of the dental X-ray imaging unit 102 may for example be captured by at least one internal imaging device 104b of the dental X-ray imaging unit 102 as similarly as at the step 610 discussed above. At a step 1320, the control system 106 may receive the scan request similarly as discussed above referring to the step 220, but the scan request may further comprise device accessory data indicating the required device accessories. At a step 1330, the control system 106 may detect one or more device accessories based on the obtained at least one optical image 105 of the patient 600 and the at least one image analysis model 118. In other words, the control system 106 may detect the one or more device accessories from the at least one optical image 105 of the patient 600 by using at least one image analysis model 1118, e.g. at least one AI-based model. The detected one or more device accessories may further be classified by using the at least one image analysis model 1118 to identify the detected one or more device accessories, i.e. whether the one or more detected device accessories belong to the required device accessories indicated in the scan request or whether the one or more detected device accessories belong to incorrect device accessories, which are not indicated as the required device accessories in the scan request. At a step 1230, in response to identifying one or more incorrect device accessories, the control system 106 may guide via one or more user interface devices e.g. via a display device and/or a loudspeaker device, the operator of the dental X-ray imaging unit 102 to remove and/or replace the one or more incorrect device accessories. The one or more user interface device may comprise the user interface device 140a and/or one or more other user interface devices.

At least some aspects of the present invention described above enable reduction of imaging time, remove at least partly a need of scout images in the CT-imaging, reduce patient dose, improve patient positioning, and/or minimizing need of additional imaging.

The specific examples provided in the description given above should not be construed as limiting the applicability and/or the interpretation of the appended claims. Lists and groups of examples provided in the description given above are not exhaustive unless otherwise explicitly stated.

1. A dental X-ray imaging system for dental X-ray imaging of a patient, the system comprising:
a dental X-ray imaging unit comprising:
an X-ray source for emitting X-rays,
an X-ray imaging detector for receiving the X-rays from the source, and
a gantry comprising the source and the imaging detector, and
a control system configured to:
obtain at least one optical image of the patient;
receive a scan request comprising region of interest (ROI) data; and
define a ROI position based on the ROI data, the at least one optical image dental atlas data, and at least one image analysis model formed based on previously collected reference image data.

2. The dental X-ray imaging system according to claim 1, wherein the at least one optical image of the patient comprises at least one optical image where a dentition of the patient is at least partly visible, and wherein the control system is configured to:
determine a plurality of head landmarks of the patient based on the at least one optical image of the patient and the at least one image analysis model,
select a plurality of atlas landmarks corresponding to the plurality of head landmarks, of the patient based on the dental atlas data, and
register the plurality of atlas landmarks and the plurality of head landmarks of the patient to determine aligned dental atlas data of the patient.

3. The dental X-ray imaging system according to claim 2, wherein the control system is configured to define the ROI position based on the ROI data and the determined aligned dental atlas data of the patient.

4. The dental X-ray imaging system according to claim 1, wherein the control system is further configured to determine exposure parameters for the scan of the patient based on the imaging mode data further comprised in the scan request, the at least one optical image of the patient and the at least one image analysis model.

5. The dental X-ray imaging system according to claim 4, wherein the control system is configured to:
determine a plurality of head landmarks of the patient based on the at least one optical image of the patient and the at least one image analysis model,
determine head size data of the patient based on the plurality of head landmarks of the patient and
use the head size data in the determination of the exposure parameters.

6. The dental X-ray imaging system according to claim 5, wherein the control system is further configured to:
determine classification data of the patient based on the at least one optical image of the patient and the at least one image analysis model, and
use the classification data in the determination of the exposure parameters.

7. The dental X-ray imaging system according to claim 1, wherein the control system is further configured to:
determine at least one head landmark of the patient based on the at least one optical image of the patient and the at least one image analysis model,
determine height data of the patient based on the at least one head landmark of the patient, and
use the determined height data to adjust a height of the parts of the dental X-ray imaging unit.

8. The dental X-ray imaging system according to claim 1, wherein the control system is further configured to:
determine a plurality of body landmarks of the patient based on the at least one optical image of the patient and the at least one image analysis model
determine width data of the patient based on the plurality of body landmarks of the patient, and
use the determined width data to reduce a collision risk between the patient and the gantry part of the dental X-ray imaging unit.

9. The dental X-ray imaging system according to claim 1, comprising at least one optical imaging device configured to capture the at least one optical image of the patient.

10. The dental X-ray imaging system according to claim 1, wherein the ROI data comprises an indication of at least one of the following: a single tooth, a range of teeth, a dental arch, both dental arches, a temporomandibular joint (TMJ), a whole dentition and the TMJs.

11. The dental X-ray imaging system according to claim 1, wherein the control system is further configured to produce patient position correction data for the patient positioning based on the at least one optical image of the patient and the at least one image analysis model.

12. A method for dental imaging, the method is performed by an X-ray dental imaging system according to claim 1, wherein the method comprises:
obtaining at least one optical image of the patient;
receiving a scan request comprising region of interest (ROI) data; and
defining a ROI position based on the ROI data, the at least one optical image, dental atlas data, and at least one image analysis model formed based on previously collected reference image data.

13. (canceled)

14. A tangible non-volatile computer-readable medium comprising instructions which, when executed by a computer, cause the computer to:
obtain at least one optical image of the patient;
receive a scan request comprising region of interest (ROI) data; and
define a ROI position based on the ROI data,
the at least one optical image, dental atlas data, and at least one image analysis model formed based on previously collected reference image data.

15. A method for determining aligned dental atlas data of a patient, the method comprising:
obtaining at least one optical image of the patient where a dentition of the patient is at least partly visible;
determining a plurality of head landmarks of the patient based on the at least one optical image of the patient and at least one image analysis model formed based on previously collected reference image data;
selecting a plurality of atlas landmarks corresponding to the plurality of head landmarks of the patient based on dental atlas data; and
registering the plurality of atlas landmarks and the plurality of head landmarks of the patient to determine the aligned dental atlas data of the patient.

16. (canceled)

17. A tangible non-volatile computer-readable medium comprising instructions which, when executed by a computer, cause the computer to:
obtain at least one optical image of the patient, where a dentition of the patient is at least partly visible;
determine a plurality of head landmarks of the patient based on the at least one optical image of the patient and at least one image analysis model formed based on previously collected reference image data;
select a plurality of atlas landmarks corresponding to the plurality of head landmarks of the patient based on dental atlas data; and
register the plurality of atlas landmarks and the plurality of head landmarks of the patient to determine the aligned dental atlas data of the patient.
